SEQUENCE LISTING (35 sequences)

SEQ ID NO: 1 - DNA, 10 bp, Artificial Sequence
  first randomized region (R1) of Aptamer 1 (Ima-C5)
ccccgctatg

SEQ ID NO: 2 - DNA, 40 bp, Artificial Sequence
  second randomized region (R2) of Aptamer 1 (Ima-C5)
gttcggtgtgtttttaaagggtacagatcctgggcggggg

SEQ ID NO: 3 - DNA, 52 bp, Artificial Sequence
  best performing minimal effective nucleic acid fragment (F6b) of Aptamer 1 (Ima-C5)
ccccgctatgtgaggctcgatcgttcggtgtgtttttaaagggtacagatcc

SEQ ID NO: 4 - DNA, 47 bp, Artificial Sequence
  nucleic acid fragment (F6a) of Ima-C5 with improved binding to Imatinib as compared to full length Ima-C5
ctatgtgaggctcgatcgttcggtgtgtttttaaagggtacagatcc

SEQ ID NO: 5 - DNA, 56 bp, Artificial Sequence
  nucleic acid fragment (F6c) of Ima-C5 with improved binding to Imatinib as compared to full length Ima-C5
ctccccccgctatgtgaggctcgatcgttcggtgtgtttttaaagggtacagatcc

SEQ ID NO: 6 - DNA, 61 bp, Artificial Sequence
  nucleic acid fragment (F6d) of Ima-C5 with improved binding to Imatinib as compared to full length Ima-C5
tttttctccccccgctatgtgaggctcgatcgttcggtgtgtttttaaagggtacagatcc

SEQ ID NO: 7 - DNA, 66 bp, Artificial Sequence
  nucleic acid fragment (F6e) of Ima-C5 with improved binding to Imatinib as compared to full length Ima-C5
cgctctttttctccccccgctatgtgaggctcgatcgttcggtgtgtttttaaagggtacagatcc

SEQ ID NO: 8 - DNA, 52 bp, Artificial Sequence
  nucleic acid fragment (F7a) of Ima-C5 with improved binding to Imatinib as compared to full length Ima-C5
ctatgtgaggctcgatcgttcggtgtgtttttaaagggtacagatcctgggc

SEQ ID NO: 9 - DNA, 57 bp, Artificial Sequence
  nucleic acid fragment (F7b) of Ima-C5 with improved binding to Imatinib as compared to full length Ima-C5
ccccgctatgtgaggctcgatcgttcggtgtgtttttaaagggtacagatcctgggc

SEQ ID NO: 10 - DNA, 61 bp, Artificial Sequence
  nucleic acid fragment (f7c) of ima-c5 with improved binding toImatinib as compared to full length Ima-C5
ctccccccgctatgtgaggctcgatcgttcggtgtgtttttaaagggtacagatcctgggc

SEQ ID NO: 11 - DNA, 66 bp, Artificial Sequence
  nucleic acid fragment (F7d) of Ima-C5 with improved binding to Imatinib as compared to full length Ima-C5
tttttctccccccgctatgtgaggctcgatcgttcggtgtgtttttaaagggtacagatcctgggc

SEQ ID NO: 12 - DNA, 71 bp, Artificial Sequence
  nucleic acid fragment (F7e) of Ima-C5 with improved binding to Imatinib as compared to full length Ima-C5
cgctctttttctccccccgctatgtgaggctcgatcgttcggtgtgtttttaaagggtacagatcctgggc

SEQ ID NO: 13 - DNA, 52 bp, Artificial Sequence; identical to SEQ ID NO: 3
  nucleic acid fragment (F14f) of Ima-C5 with improved binding to Imatinib as compared to full length Ima-C5

SEQ ID NO: 14 - DNA, 57 bp, Artificial Sequence; identical to SEQ ID NO: 9
  nucleic acid fragment (F14g) of Ima-C5 with improved binding to Imatinib as compared to full length Ima-C5

SEQ ID NO: 15 - DNA, 62 bp, Artificial Sequence
  nucleic acid fragment (F14h) of Ima-C5 with improved binding to Imatinib as compared to full length Ima-C5
ccccgctatgtgaggctcgatcgttcggtgtgtttttaaagggtacagatcctgggcggggg

SEQ ID NO: 16 - DNA, 62 bp, Artificial Sequence; identical to SEQ ID NO: 15
  nucleic acid fragment (F14i) of Ima-C5 with improved binding to Imatinib as compared to full length Ima-C5

SEQ ID NO: 17 - DNA, 67 bp, Artificial Sequence
  nucleic acid fragment (F14j) of Ima-C5 with improved binding to Imatinib as compared to full length Ima-C5
ccccgctatgtgaggctcgatcgttcggtgtgtttttaaagggtacagatcctgggcggggggcatt

SEQ ID NO: 18 - DNA, 72 bp, Artificial Sequence
  nucleic acid fragment (F14k) of Ima-C5 with improved binding to Imatinib as compared to full length Ima-C5
ccccgctatgtgaggctcgatcgttcggtgtgtttttaaagggtacagatcctgggcggggggcattgaggg

SEQ ID NO: 19 - DNA, 77 bp, Artificial Sequence
  nucleic acid fragment (F14l) of Ima-C5 with improved binding to Imatinib as compared to full length Ima-C5
ccccgctatgtgaggctcgatcgttcggtgtgtttttaaagggtacagatcctgggcggggggcattgagggtgaca

SEQ ID NO: 20 - DNA, 71 bp, Artificial Sequence
  nucleic acid fragment (F6) of Aptamer 1 (Ima-C5)
atccacgctctttttctccccccgctatgtgaggctcgatcgttcggtgtgtttttaaagggtacagatcc

SEQ ID NO: 21 - DNA, 76 bp, Artificial Sequence
  nucleic acid fragment (F7) of Aptamer 1 (Ima-C5)
atccacgctctttttctccccccgctatgtgaggctcgatcgttcggtgtgtttttaaagggtacagatcctgggc

SEQ ID NO: 22 - DNA, 81 bp, Artificial Sequence
  nucleic acid fragment (F8) of Aptamer 1 (Ima-C5)
atccacgctctttttctccccccgctatgtgaggctcgatcgttcggtgtgtttttaaagggtacagatcctgggcggggg

SEQ ID NO: 23 - DNA, 81 bp, Artificial Sequence
  nucleic acid fragment (F14) of Aptamer 1 (Ima-C5)
ccccgctatgtgaggctcgatcgttcggtgtgtttttaaagggtacagatcctgggcggggggcattgagggtgacatagg

SEQ ID NO: 24 - DNA, 100 bp, Artificial Sequence
  full nucleic acid sequence of Aptamer 1 (Ima-C5)
atccacgctctttttctccccccgctatgtgaggctcgatcgttcggtgtgtttttaaagggtacagatcctgggcggggggcattgagggtgacatagg

SEQ ID NO: 25 - DNA, 10 bp, Artificial Sequence
  first randomized region (R1) of Aptamer 2 (Ima-E8)
gtggactaga

SEQ ID NO: 26 - DNA, 40 bp, Artificial Sequence
  second randomized region (R2) of Aptamer 2 (Ima-E8)
tacttaatcatgttaagagtccacgtcttgagttgtggat

SEQ ID NO: 27 - DNA, 86 bp, Artificial Sequence
  nucleic acid fragment (F10) of Aptamer 2 (Ima-E8)
atccacgctctttttctccgtggactagatgaggctcgatctacttaatcatgttaagagtccacgtcttgagttgtggatgcatt

SEQ ID NO: 28 - DNA, 91 bp, Artificial Sequence
  nucleic acid fragment (F11) of Aptamer 2 (Ima-E8)
atccacgctctttttctccgtggactagatgaggctcgatctacttaatcatgttaagagtccacgtcttgagttgtggatgcattgaggg

SEQ ID NO: 29 - DNA, 96 bp, Artificial Sequence
  nucleic acid fragment (F12) of Aptamer 2 (Ima-E8)
atccacgctctttttctccgtggactagatgaggctcgatctacttaatcatgttaagagtccacgtcttgagttgtggatgcattgagggtgaca

SEQ ID NO: 30 - DNA, 100 bp, Artificial Sequence
  full nucleic acid sequence of Aptamer 2 (Ima-E8)
atccacgctctttttctccgtggactagatgaggctcgatctacttaatcatgttaagagtccacgtcttgagttgtggatgcattgagggtgacatagg

SEQ ID NO: 31 - DNA, 12 bp, Artificial Sequence
  exemplary immobilisation region (I)
tgaggctcgatc

SEQ ID NO: 32 - DNA, 19 bp, Artificial Sequence
  exemplary first primer region (P1)
atccacgctctttttctcc

SEQ ID NO: 33 - DNA, 19 bp, Artificial Sequence
  exemplary second primer region (P2)
gcattgagggtgacatagg

SEQ ID NO: 34 - DNA, 12 bp, Artificial Sequence
  exemplary immobilisation sequence
gatcgagcctca

SEQ ID NO: 35 - DNA, 19 bp, Artificial Sequence
  exemplary reverse second primer region (P2)
cctatgtcaccctcaatgc

(19) United States
(12) Patent Application Publication
(10) Pub. No.: US 2022/0112499 A1
(43) Pub. Date: Apr. 14, 2022

(54) APTAMERS AGAINST IMATINIB

(71) Applicant: APTAMER DIAGNOSTICS LIMITED, North Yorkshire (GB)

(72) Inventors: Christine REINEMANN, North Yorkshire (GB); Edward BARNES, North Yorkshire (GB); David Harry John BUNKA, North Yorkshire (GB); Arron Craig TOLLEY, North Yorkshire (GB)

(21) Appl. No.: 17/297,599

(22) PCT Filed: Nov. 27, 2019
(86) PCT No.: PCT/GB2019/053353

§ 371 Date: May 27, 2021

(30) Foreign Application Priority Data
Nov. 30, 2018 (GB) 1819580.0

(51) Int. Cl. C12N 15/115; C12Q 1/6837; A61K 31/506; C12N 15/10

(52) U.S. Cl. CPC C12N 15/115; C12Q 1/6837; C12N 2320/13; C12N 15/1048; C12N 2310/16; A61K 31/506

(57) ABSTRACT

The present invention relates inter alia to aptamers that specifically bind to Imatinib and methods of using the same.

### FIELD OF THE INVENTION

Embodiments of the present invention relate to aptamers that specifically bind to Imatinib and methods of using the same. For example, certain embodiments of the invention relate to methods of detecting the presence, absence or amount of Imatinib in a sample using the aptamers described herein.

### BACKGROUND TO THE INVENTION

Imatinib, a 2-phenyl amino pyrimidine derivative, is a tyrosine kinase inhibitor with activity against ABL, BCR-ABL, PDGFRA and c-KIT. Imatinib binds close to the ATP binding site of such targets, inhibiting enzyme activity and downstream signalling pathways that promote oncogenesis.

Imatinib is an oral targeted therapy used to treat cancer, especially leukaemia or blood disorders. For example, Imatinib is used as a first line therapy in the treatment of chronic myeloid leukaemia (CML). Pharmacokinetic studies have shown considerable variability in trough concentrations of Imatinib due to variations in its metabolism, poor compliance, or drug-drug interactions. As plasma levels of Imatinib are correlated with response to therapy, monitoring of the therapeutic levels of the drug (and adjusting to the required target levels) would be of value in increasing efficacy and minimizing toxicity.

Imatinib is a low-molecular weight drug (C₂₉H₃₁N₇O, average molecular weight 493.6027 Da) hindering efforts to develop immunoassays using specific antibodies that do not cross-react with the drug's metabolites. For example, small molecules make very poor targets for affinity reagents. Typically, small molecules feature a very low number of functional groups and therefore affinity reagents struggle to bind specifically to such substrates. Moreover, small molecules may have toxicity issues and/or lack of immunogenicity. Despite these issues, there remains a need to develop agents which are simpler and easier to adapt to assay platforms, are more reliable to produce, do not rely on a pair of affinity ligands and give a gain-of-signal readout; whilst being capable of specifically binding to Imatinib and its pharmacologically active salts without any cross-reactivity with closely related compounds or drug metabolites.

Imatinib levels in the serum of CML patients have been evaluated using chromatographic techniques such as liquid chromatography coupled with mass spectrometry or ultraviolet spectrophotometry detection (Micova et al. Clin Chim Acta. 2010; 411; 1957-62). However, such techniques are costly, time-consuming and require specialist laboratories, expensive equipment, heavy use of biological material, solvents and other materials.

In the case of imatinib, several antibody based tests have been developed, but these all have the same limitations associated with small molecule targeting immunoassays; they rely on a pair of antibodies (which can be difficult to isolate and expensive to produce) and/or rely on a competitive assay format which uses a ‘loss-of-signal’ output. Competitive assays of this nature are known to be prone to high background signals and a lack of sensitivity.

It is an aim of some embodiments of the present invention to at least partially mitigate some of the problems identified in the prior art by developing detection agents which are more reliable to produce, do not rely on a pair of affinity ligands and give a gain-of-signal readout as compared to antibody-based tests.

### SUMMARY OF CERTAIN EMBODIMENTS OF THE INVENTION

The present invention relates to the development of Imatinib binding aptamers and methods of using the same.

The aptamers described herein are shown to work effectively and provide a simple means of testing the presence, absence or amount of Imatinib in a sample using a simple gain-of signal assay format. In particular, the aptamers described herein are capable of binding to Imatinib with high affinity. The aptamers described herein allow clinical ranges of active Imatinib (i.e. less than 1 μM) to be detected in biological fluids.

Accordingly, certain aspects of the present invention provide inter alia:
an aptamer capable of specifically binding to Imatinib, wherein the aptamer comprises or consists of:
(a) a nucleic acid sequence selected from any one of SEQ ID NOs: 3 to 24 or 27 to 30;
(b) a nucleic acid sequence having at least 85% identity with any one of SEQ ID NOs: SEQ ID NOs: 3 to 24 or 27 to 30;
(c) a nucleic acid sequence having at least about 30 consecutive nucleotides of any one of SEQ ID NOs 3 to 24 or 27 to 30; or
(d) a nucleic acid sequence having at least about 30 consecutive nucleotides of a sequence having at least 85% identity with any one of SEQ ID Nos 3 to 24 or 27 to 30;
an aptamer that competes for binding to Imatinib with the aptamers as described herein;
a complex comprising any aptamer as described herein and a detectable molecule;
a biosensor or test strip comprising any aptamer as described herein.
apparatus for detecting the presence, absence or level of Imatinib in a sample, the apparatus comprising:
   (i) a support; and
   (ii) any aptamer as described herein;
use of any aptamer, complex, biosensor, test strip and/or apparatus as described herein for detecting, enriching, separating and/or isolating Imatinib.
methods of detecting the presence, absence or amount of Imatinib in a sample, the method comprising:
   (i) interacting the sample with any aptamer described herein; and
   (ii) detecting the presence, absence or amount of Imatinib.
methods of treating or preventing cancer in a subject, the method comprising:
(i) administering an initial dose of Imatinib to the subject;
(ii) detecting the amount of Imatinib in a sample obtained from the subject according to any method described herein; and
(iii) (a) if the level of Imatinib is below a lower threshold level, administering an increased dose of Imatinib to the subject;
   (b) is the level of Imatinib is above an upper threshold level, administering a decreased dose of Imatinib to the subject.
kits for detecting and/or quantifying Imatinib, the kit comprising any aptamer as described herein.

### DETAILED DESCRIPTION OF CERTAIN EMBODIMENTS OF THE INVENTION

### Brief Description of the Figures

Certain embodiments of the present invention will be described in more detail below, with reference to the accompanying Figures in which:

FIG. 1 shows the predicted secondary structure of aptamers against Imatinib (A—aptamer 1 (Ima-C5) B—aptamer 2 (Ima-E8). Secondary structures were determined using Mfold [Zuker, M. (2003) Mfold web server for nucleic acid folding and hybridization prediction. Nucleic Acid Res. 31(13), 3406-15]. The binding site for the immobilisation oligonucleotide is highlighted in green.

FIG. 2 shows the progressive increase in fluorescence from recovered aptamers in successive rounds of selection as the aptamer library becomes enriched. After round 10, a target specific polyclonal population was isolated.

FIG. 3 shows model data for a ‘dip and read’ Biolayer Interferometry (BLI) assay, used to monitor aptamer binding to its target, identify the best performing aptamer clones and for kinetic analysis. The model data shows the ‘Immobilisation of aptamers’ onto the sensor surface, establishment of a new baseline during ‘Washing’ and a subsequent reduction in signal as aptamers bind to their target and ‘Dissociate’ from the sensor surface.

FIG. 4 shows binding of the polyclonal aptamer population monitored using the BLI assay. Data shows only the ‘dissociation by target binding’ described in FIG. 3 and has been background subtracted and ‘flipped’ to allow the use of the software Steady State Analysis algorithm. BLI assays show improvement in binding of the selected aptamer population to the selection target Imatinib (red) and its main metabolite N-desmethyl Imatinib (green) in comparison to the starting library (blue).

FIG. 5 shows BLI assay data used for ‘hit picking’ of the best performing monoclonal aptamers. Data shows only the ‘dissociation by target binding’ described in FIG. 3, and has been background subtracted and ‘flipped’. Results show identification of aptamers with improved binding to Imatinib, relative to other screened sequences. Two aptamers have high affinity to Imatinib as compared to the other enriched aptamer population from selection round 10.

FIG. 6 shows comparative binding studies to determine aptamer specificity. Aptamer 1 (Ima-C5) and Aptamer 2 (Ima-C8) showed improved binding to Imatinib (red trace) and its main metabolite (green trace) relative to both the starting library and the enriched aptamer population from selection round 10. Other tested molecules (structurally and functionally related) were not bound (blue and purple traces). Specificity studies were carried out using the BLI assay described in FIG. 3. Data shows only the ‘dissociation by target binding’ described in FIG. 3 and has been background subtracted and ‘flipped’.

FIG. 7 shows Aptamer Ima C5 binding to the target Imatinib in a concentration dependent manner. Imatinib interaction was monitored by surface plasmon resonance (SPR) using a direct binding assay in which Aptamer Ima C5 was immobilised onto a sensor chip in a Biacore instrument. This was then interacted with a concentration gradient of Imatinib. The Affinity constant (K_{D} value) was calculated using Biacore Insight evaluation software with a 1:1 binding Langmuir binding model with local RI parameter. The affinity of aptamer 1 to Imatinib (in PBS6) was calculated with 1.10×10⁻⁷ M (110 nM).

FIG. 8 shows the use of Aptamer 1 in an ELISA-like assay format, binding to the target in buffered human plasma. Functionality of the best performing aptamer (Aptamer 1) was demonstrated using microtiter plate-based aptamer displacement assay (fluorescence assay). The selected aptamer shows strong, concentration dependent binding to its target Imatinib (leading to a gain-of-signal response) in the presence of different concentrations of human plasma, with minimal background binding to the plasma alone. Assays were carried out at target concentrations that reflect the therapeutic range of Imatinib.

FIG. 9 shows BLI displacement assay binding studies used to identify the minimal effective fragments of Aptamer 1. A panel of truncated versions of Aptamer 1 was tested for binding to target Imatinib (10 μM in PBS6). The smallest and best performing fragment of Aptamer 1 is identified herein as SEQ ID NO: 3 (Ima-C5-F6b, red binding curve). Minimal fragment identification studies were carried out using the BLI assay described in FIG. 3. Data shows only the ‘dissociation by target binding’ described in FIG. 3 and has been background subtracted and ‘flipped’.

FIG. 10 shows aptamer fragment Ima C5-F6b binding to the target Imatinib in a concentration dependent manner. Imatinib interaction was monitored by surface plasmon resonance (SPR) using a direct binding assay in which Aptamer fragment Ima C5-F6b was immobilised onto a sensor chip in a Biacore instrument. This was then interacted with a concentration gradient of Imatinib. The Affinity constant (K_{D} value) was calculated using Biacore Insight evaluation software with a 1:1 binding Langmuir binding model with local RI parameter. The affinity of aptamer fragment Ima C5-F6b to Imatinib (in PBS6) was calculated with 7.21×10⁻⁸ M (72.1 nM).

FIG. 11 shows BLI based displacement assay binding studies used to determine the specificity of Aptamer fragment Ima C5-F6b. Binding curves showing aptamer binding to Imatinib (red, 10 μM), the metabolite N-desmethyl Imatinib (green, 10 μM) and negative target, Irinotecan (purple, 10 μM). Specificity studies were carried out using the BLI assay described in FIG. 3. Data shows only the ‘dissociation by target binding’ described in FIG. 3 and has been background subtracted and ‘flipped’.

FIG. 12 shows the use of aptamer Ima C5-F6b in an ELISA-like assay format, binding to the target in buffered human plasma. Functionality of Ima C5-F6b was tested using microtiter plate-based Aptamer Displacement assay (fluorescence assay). The selected aptamer shows strong, concentration dependent binding to its target Imatinib (leading to a gain-of-signal response) in the presence of different concentrations of human plasma, with minimal background binding to the plasma alone. Tests were carried out at target concentrations that reflect the therapeutic range of this drug.

Sequence listing
SEQ ID NO: 1 shows a first randomized region (R1) of Aptamer 1 (Ima-C5)
CCCCGCTATG
SEQ ID NO: 2 shows a second randomized region (R2) of Aptamer 1 (Ima-C5)
GTTCGGTGTGTTTTTAAAGGGTACAGATCCTGGGCGGGGG
SEQ ID NO: 3 shows the best performing minimal effective nucleic acid fragment (F6b) of
Aptamer 1 (Ima-C5)
CCCCGCTATGTGAGGCTCGATCGTTCGGTGTGTTTTTAAAGGGTACAGATCC
SEQ ID NO: 4 shows a nucleic acid fragment (F6a) of Ima-C5 with improved binding to Imatinib
as compared to full length Ima-C5
CTATGTGAGGCTCGATCGTTCGGTGTGTTTTTAAAGGGTACAGATCC
SEQ ID NO: 5 shows a nucleic acid fragment (F6c) of Ima-C5 with improved binding to Imatinib
as compared to full length Ima-C5
CTCCCCCCGCTATGTGAGGCTCGATCGTTCGGTGTGTTTTTAAAGGGTACAGATCC
SEQ ID NO: 6 shows a nucleic acid fragment (F6d) of Ima-C5 with improved binding to Imatinib
as compared to full length Ima-C5
TTTTTCTCCCCCCGCTATGTGAGGCTCGATCGTTCGGTGTGTTTTTAAAGGGTACAGAT
CC
SEQ ID NO: 7 shows a nucleic acid fragment (F6e) of Ima-C5 with improved binding to Imatinib
as compared to full length Ima-C5
CGCTCTTTTTCTCCCCCCGCTATGTGAGGCTCGATCGTTCGGTGTGTTTTTAAAGGGTA
CAGATCC
SEQ ID NO: 8 shows a nucleic acid fragment (F7a) of Ima-C5 with improved binding to Imatinib
as compared to full length Ima-C5
CTATGTGAGGCTCGATCGTTCGGTGTGTTTTTAAAGGGTACAGATCCTGGGC
SEQ ID NO: 9 shows a nucleic acid fragment (F7b) of Ima-C5 with improved binding to Imatinib
as compared to full length Ima-C5
CCCCGCTATGTGAGGCTCGATCGTTCGGTGTGTTTTTAAAGGGTACAGATCCTGGGC
SEQ ID NO: 10 shows a nucleic acid fragment (F7c) of Ima-C5 with improved binding to
Imatinib as compared to full length Ima-C5
CTCCCCCCGCTATGTGAGGCTCGATCGTTCGGTGTGTTTTTAAAGGGTACAGATCCTG
GGC
SEQ ID NO: 11 shows a nucleic acid fragment (F7d) of Ima-C5 with improved binding to
Imatinib as compared to full length Ima-C5
TTTTTCTCCCCCCGCTATGTGAGGCTCGATCGTTCGGTGTGTTTTTAAAGGGTACAGAT
CCTGGGC
SEQ ID NO: 12 shows a nucleic acid fragment (F7e) of Ima-C5 with improved binding to
Imatinib as compared to full length Ima-C5
CGCTCTTTTTCTCCCCCCGCTATGTGAGGCTCGATCGTTCGGTGTGTTTTTAAAGGGTA
CAGATCCTGGGC
SEQ ID NO: 13 shows a nucleic acid fragment (F14f) of Ima-C5 with improved binding to
Imatinib as compared to full length Ima-C5
CCCCGCTATGTGAGGCTCGATCGTTCGGTGTGTTTTTAAAGGGTACAGATCC
SEQ ID NO: 14 shows a nucleic acid fragment (F14g) of Ima-C5 with improved binding to
Imatinib as compared to full length Ima-C5
CCCCGCTATGTGAGGCTCGATCGTTCGGTGTGTTTTTAAAGGGTACAGATCCTGGGC
SEQ ID NO: 15 shows a nucleic acid fragment (F14h) of Ima-C5 with improved binding to
Imatinib as compared to full length Ima-C5
CCCCGCTATGTGAGGCTCGATCGTTCGGTGTGTTTTTAAAGGGTACAGATCCTGGGCG
GGGG
SEQ ID NO: 16 shows a nucleic acid fragment (F14i) of Ima-C5 with improved binding to
Imatinib as compared to full length Ima-C5
CCCCGCTATGTGAGGCTCGATCGTTCGGTGTGTTTTTAAAGGGTACAGATCCTGGGCG
GGGG
SEQ ID NO: 17 shows a nucleic acid fragment (F14j) of Ima-C5 with improved binding to
Imatinib as compared to full length Ima-C5
CCCCGCTATGTGAGGCTCGATCGTTCGGTGTGTTTTTAAAGGGTACAGATCCTGGGCG
GGGGGCATT
SEQ ID NO: 18 shows a nucleic acid fragment (F14k) of Ima-C5 with improved binding to
Imatinib as compared to full length Ima-C5
CCCCGCTATGTGAGGCTCGATCGTTCGGTGTGTTTTTAAAGGGTACAGATCCTGGGCG
GGGGGCATTGAGGG
SEQ ID NO: 19 shows a nucleic acid fragment (F14I) of Ima-C5 with improved binding to
Imatinib as compared to full length Ima-C5
CCCCGCTATGTGAGGCTCGATCGTTCGGTGTGTTTTTAAAGGGTACAGATCCTGGGCG
GGGGGCATTGAGGGTGACA
SEQ ID NO: 20 shows a nucleic acid fragment (F6) of Aptamer 1 (Ima-C5)
ATCCACGCTCTTTTTCTCCCCCCGCTATGTGAGGCTCGATCGTTCGGTGTGTTTTTAAA
GGGTACAGATCC
SEQ ID NO: 21 shows a nucleic acid fragment (F7) of Aptamer 1 (Ima-C5)
ATCCACGCTCTTTTTCTCCCCCCGCTATGTGAGGCTCGATCGTTCGGTGTGTTTTTAAA
GGGTACAGATCCTGGGC
SEQ ID NO: 22 shows a nucleic acid fragment (F8) of Aptamer 1 (Ima-C5)
ATCCACGCTCTTTTTCTCCCCCCGCTATGTGAGGCTCGATCGTTCGGTGTGTTTTTAAA
GGGTACAGATCCTGGGCGGGGG
SEQ ID NO: 23 shows a nucleic acid fragment (F14) of Aptamer 1 (Ima-C5)
CCCCGCTATGTGAGGCTCGATCGTTCGGTGTGTTTTTAAAGGGTACAGATCCTGGGCG
GGGGGCATTGAGGGTGACATAGG
SEQ ID NO: 24 shows the full nucleic acid sequence of Aptamer 1 (Ima-C5)
ATCCACGCTCTTTTTCTCCCCCCGCTATGTGAGGCTCGATCGTTCGGTGTGTTTTTAAA
GGGTACAGATCCTGGGCGGGGGGCATTGAGGGTGACATAGG
SEQ ID NO: 25 shows a first randomized region (R1) of Aptamer 2 (Ima-E8)
GTGGACTAGA
SEQ ID NO: 26 shows a second randomized region (R2) of Aptamer 2 (Ima-E8)
TACTTAATCATGTTAAGAGTCCACGTCTTGAGTTGTGGAT
SEQ ID NO: 27 shows a nucleic acid fragment (F10) of Aptamer 2 (Ima-E8)
ATCCACGCTCTTTTTCTCCGTGGACTAGATGAGGCTCGATCTACTTAATCATGTTAAGAG
TCCACGTCTTGAGTTGTGGATGCATT
SEQ ID NO: 28 shows a nucleic acid fragment (F11) of Aptamer 2 (Ima-E8)
ATCCACGCTCTTTTTCTCCGTGGACTAGATGAGGCTCGATCTACTTAATCATGTTAAGAG
TCCACGTCTTGAGTTGTGGATGCATTGAGGG
SEQ ID NO: 29 shows a nucleic acid fragment (F12) of Aptamer 2 (Ima-E8)
ATCCACGCTCTTTTTCTCCGTGGACTAGATGAGGCTCGATCTACTTAATCATGTTAAGAG
TCCACGTCTTGAGTTGTGGATGCATTGAGGGTGACA
SEQ ID NO: 30 shows the full nucleic acid sequence of Aptamer 2 (Ima-E8)
ATCCACGCTCTTTTTCTCCGTGGACTAGATGAGGCTCGATCTACTTAATCATGTTAAGAG
TCCACGTCTTGAGTTGTGGATGCATTGAGGGTGACATAGG
SEQ ID NO: 31 shows an exemplary immobilisation region (I)
TGAGGCTCGATC
SEQ ID NO: 32 shows an exemplary first primer region (P1)
ATCCACGCTCTTTTTCTCC
SEQ ID NO: 33 shows an exemplary second primer region (P2)
GCATTGAGGGTGACATAGG
SEQ ID NO: 34 shows an exemplary immobilisation sequence
GATCGAGCCTCA
SEQ ID NO: 35 shows an exemplary reverse second primer region (P2)
CCTATGTCACCCTCAATGC

As explained further below, any underlined sequence refers to first (P1) and second (P2) primer sites and any italic sequence refers to the immobilisation region (I) of the aptamer (i.e., nucleic acid sequence of the aptamer capable of binding to at least a portion of immobilisation sequence). R1 and R2 refer to first and second randomized regions respectively.

### DETAILED DESCRIPTION

Further features of certain embodiments of the present invention are described below. The practice of embodiments of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y. Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure.

Units, prefixes and symbols are denoted in their Systeme International de Unitese (SI) accepted form. Numeric ranges are inclusive of the numbers defining the range. Unless otherwise indicated, amino acid sequences are written left to right in amino to carboxy orientation and nucleic acid sequences are written left to right in 5′ to 3′ orientation.

In the following, the invention will be explained in more detail by means of non-limiting examples of specific embodiments. In the example experiments, standard reagents and buffers free from contamination are used.

### Imatinib

The invention provides aptamers capable of specifically binding to Imatinib.

Imatinib has the following structure:

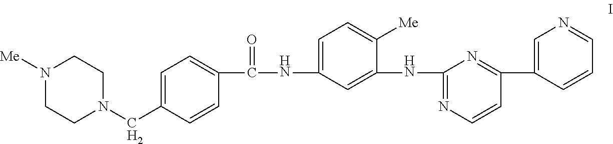 [SMILES: Cc1ccc(NC(=O)c2ccc(CN3CCN(C)CC3)cc2)cc1Nc1nccc(-c2cccnc2)n1]

Imatinib and its salts (e.g. Imatinib mesylate) are used to treat cancer. For example, Imatinib may be used to treat chronic myelogenous leukemia (CML) and acute lymphocytic leukemia (ALL) that are Philadelphia chromosome positive (Ph+) and certain types of gastrointestinal stromal tumors (GIST), systemic mastocytosis and myelodysplastic syndrome.

Typically, Imatinib is taken orally. Common side effects include vomiting, diarrhea, muscle pain, headache and rash. Severe side effects include fluid retention, gastrointestinal bleeding, bone marrow suppression, liver problems and heart failure.

The preferred pharmacologically active salt of Imatinib is Imatinib mesylate, and has the structure:

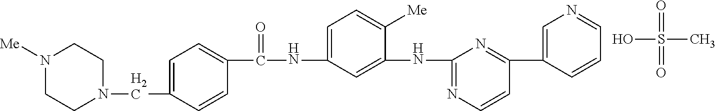

The aptamers of the invention bind specifically to Imatinib and/or its pharmacologically active salts. In certain embodiments, the aptamers of the invention bind specifically to Imatinib mesylate.

In certain embodiments, the aptamers of the invention bind specifically to pharmacologically active metabolites of Imatinib. The major active metabolite of Imatinib, N-desmethyl Imatinib, has the structure:

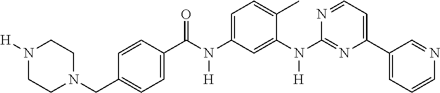 [SMILES: Cc1ccc(NC(=O)c2ccc(CN3CCNCC3)cc2)cc1Nc1nccc(-c2cccnc2)n1]

N-desmethyl Imatinib has the same in vitro potency against the Bcr-ABL kinase as Imatinib and is usually present in plasma at 10-15% of the levels of Imatinib. In certain embodiments, the aptamers bind specifically to N-desmethyl Imatinib.

As used herein, the term “Imatinib” is understood to include Imatinib and/or any of its pharmacologically active salts or metabolites, including Imatinib mesylate and/or N-desmethyl Imatinib.

An aptamer binds “specifically” to Imatinib, is an aptamer that binds with preferential or high affinity to Imatinib but does not bind or binds with only low affinity to other structurally related small molecules (e.g. Irinotecan).

In certain embodiments, an aptamer binds to Imatinib (and/or its salts) with a binding dissociation equilibrium constant (K_{D}) of less than about 1 μM, less than about 500 nM, less than about 400 nM, less than about 300 nM, less than about 200 nM, less than about 100 nM, less than about 90 nM, less than about 80 nM, less than about 70 nM or less. Binding affinity of aptamers may be measured by any method known to person skilled in the art, including, for example, surface plasmon resonance (SPR), Biolayer Interferometry (BLI), displacement assay and/or steady state analysis.

In certain embodiments, an aptamer that does not bind specifically to Imatinib is an aptamer that binds with non-preferential or low affinity to Imatinib. For example, an aptamer that binds with only low affinity to Imatinib (or with low affinity to other structurally related small molecules) may be an aptamer with a K_{D} of more than about 1 μM, more than about 2 μM, more than about 3 μM, more than about 4 μM, more than about 5 μM or more.

### Aptamers

The aptamers described herein are small artificial ligands, comprising DNA, RNA or modifications thereof, capable of specifically binding to Imatinib with high affinity and specificity.

As used herein, “aptamer”, “nucleic acid molecule” or “oligonucleotide” are used interchangeably to refer to a non-naturally occurring nucleic acid molecule that has a desirable action on a target molecule (i.e., Imatinib).

The aptamers of the invention may be DNA aptamers. For example, the aptamers may be formed from single-stranded DNA (ssDNA). Alternatively, the aptamers of the invention may be RNA aptamers. For example, the aptamers can be formed from single-stranded RNA (ssRNA). The aptamers of the invention may comprise modified nucleic acids as described herein.

In certain embodiments, the aptamers of the invention are prepared using principles of in vitro selection known in the art, that include iterative cycles of target binding, partitioning and preferential amplification of target binding sequences.

In certain embodiments, the aptamers are selected from a nucleic acid molecule library such as a single-stranded DNA or RNA nucleic acid molecule library. Typically, the aptamers are selected from a “universal aptamer selection library” that is designed such that any selected aptamers need little to no adaptation to convert into any of the listed assay formats. In certain embodiments, the “universal aptamer selection library” comprises the following functional parts: a first primer region, at least one immobilisation region, at least one randomised region and a second primer region.

In certain embodiments, the nucleotide sequences of the aptamer library have the following structure (in a 5′ to 3′ direction):

P1-R1-I-R2-P2,

wherein P1 is the first primer region, R1 is the first randomized region, I is the immobilisation region, R2 is the further randomized region and P2 is the further primer region, wherein at least R1 and/or R2 or a portion thereof are involved in target molecule binding.

Once selected, the aptamer may be further modified before being used e.g. to remove one or both primer sequences and/or parts of the randomised or immobilisation region not required for target binding.

Typically, aptamers of the invention comprise an immobilisation region (i.e., docking sequence). The immobilisation region of the aptamer may hybridise over at least a portion of an “immobilisation oligonucleotide”. Typically, the immobilisation region is complementary to at least a portion of an immobilisation oligonucleotide. Typically, the immobilisation region is between about 10 to about 20 nucleotides in length, e.g. 10, 11, 12, 13, 14, 15, 16, 17, 18, 19 or 20 nucleotides in length.

The terms “hybridises” and “hybridisation” as used herein mean to form an interaction based on Watson-crick base pairing between a fixed region within the aptamer and a complimentary sequence within the ‘immobilisation oligonucleotide’, under conventional hybridization conditions, preferably under stringent conditions, as described, for example, in Sambrook et al., Molecular Cloning, A Laboratory Manual, 3. Ed. (2001) Cold Spring Harbor Laboratory Press, Cold Spring Harbor, N.Y.

The skilled person would understand the immobilisation region of the aptamer may be selected, depending, for example, on the starting library and/or aptamer selection protocol. A variety of combinatorial random libraries are available via commercial sources. In certain embodiments, the immobilisation region comprises SEQ ID NO: 31 and/or the immobilisation oligonucleotide comprises SEQ ID NO:34.

Typically, aptamers of the invention comprise a first primer region (e.g. at the 5′ end), a second primer region (e.g. at the 3′ end), or both. The primer regions may serve as primer binding sites for PCR amplification of the library and selected aptamers.

The skilled person would understand different primer sequences can be selected depending, for example, on the starting library and/or aptamer selection protocol. For example, aptamers of the invention may comprise SEQ ID NO: 32 and/or 33.

The first primer region and/or second region may comprise a detectable label as described herein. For example, the first and/or second primer region may be fluorescently (e.g. FAM)-labelled. In certain embodiments, the first and/or second primer region primer are phosphate (PO₄) labelled.

The aptamers of the invention may be selected from a nucleic acid molecule library having a first randomized region (R1) and/or second randomized region (R2). The aptamers of the invention may comprise at least a portion of R1 and/or R2. In certain embodiments, the aptamers of the invention comprise at least a portion (e.g., at least 8 consecutive nucleotides or more) of SEQ ID NO: 1 or SEQ ID NO: 25 and/or at least a portion (e.g., at least 8 consecutive nucleotides or more) of SEQ ID NO: 2 or SEQ ID NO: 26. In certain embodiments, the aptamers of the invention comprise SEQ ID NO: 1 or SEQ ID NO: 25. In certain embodiments, the aptamers of the invention comprise at least 30 consecutive nucleotides or more of SEQ ID NO: 2 or SEQ ID NO: 26.

In certain embodiments, the aptamers of the invention comprise or consist of a nucleic acid sequence selected from any one of SEQ ID NOs: 3 to 24 or 27 to 30 (e.g. relating to the “Ima-C5” and/or “Ima-E8” aptamers).

In certain embodiments, the aptamers of the invention comprise or consist of a nucleic acid sequence selected from any one of SEQ ID NOs: 3 to 24 (e.g. relating to the “Ima-C5” aptamer).

In certain embodiments, the aptamers of the invention comprise or consist of any one of SEQ ID NOs: 3 to 19. These sequences relate to Ima-C5 fragments shown to have improved binding to Imatinib as compared to full-length Ima-C5. In certain embodiments, the aptamers of the invention comprise or consist of SEQ ID NO: 3. This minimal effective fragment is shown herein as the best performing aptamer against Imatinib.

In certain embodiments, aptamers of the invention comprise or consist of a nucleic acid sequence having at least 85%, at least 90%, at least 95%, at least 96%, at least 97%, at least 98%, at least 99% or more sequence identity to the nucleotide sequence of any one of SEQ ID NOs: 3 to 24 or 27 to 30.

In certain embodiments, the aptamers of the invention comprise or consist of a nucleic acid sequence having at least 85%, at least 90%, at least 95%, at least 96%, at least 97%, at least 98%, at least 99% or more sequence identity to any one of SEQ ID NOs: 3 to 24.

In certain embodiments, the aptamers of the invention comprise or consist of a nucleic acid sequence having at least 85%, at least 90%, at least 95%, at least 96%, at least 97%, at least 98%, at least 99% or more sequence identity to any one of SEQ ID NOs 3 to 19.

In certain embodiments, the aptamers of the invention comprise or consist of a nucleic acid sequence having at least 85%, at least 90%, at least 95%, at least 96%, at least 97%, at least 98%, at least 99% or more sequence identity to SEQ ID NO: 3.

As used herein, “sequence identity” refers to the percentage of nucleotides in a candidate sequence that are identical with the nucleotides in said sequences after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, CLUSTALW or Megalign (DNASTAR) software. For example, % nucleic acid sequence identity values can be generated using sequence comparison computer programs found on the European Bioinformatics Institute website (http://www.ebi.ac.uk).

In certain embodiments, aptamers of the invention comprise or consist of a minimal effective fragment of SEQ ID NO: 24 (full-length Ima-C5) or SEQ ID NO: 30 (full-length Ima-C8). Herein, a “minimal effective fragment” is understood to mean a fragment (e.g. portion) of the full-length aptamer (e.g. SEQ ID NO: 24 or 30 capable of binding to Imatinib with the same or improved affinity as compared to the full-length aptamer. A minimal effective fragment may compete for binding to Imatinib with the full-length aptamer (e.g. SEQ ID NO: 24 or SEQ ID NO: 30).

In certain embodiments, aptamers of the invention comprise or consist of a nucleic acid sequence comprising at least about 30, 35, 40, 45, 50, 51, 52, 53, 54, 55, 60 or more consecutive nucleotides of a sequence having at least 85%, at least 90%, at least 95%, at least 96%, at least 97%, at least 98%, at least 99% or more identity with any of SEQ ID NOs: 3 to 24 or 27 to 30. In this context the term “about” typically means the referenced nucleotide sequence length plus or minus 10% of that referenced length.

In certain embodiments, aptamers of the invention comprise or consist of a nucleic acid sequence comprising at least about 30, 35, 40, 45, 50, 51, 52, 53, 54, 55, 60 or more consecutive nucleotides of a sequence having at least 85%, at least 90%, at least 95%, at least 96%, at least 97%, at least 98%, at least 99% or more identity with any of SEQ ID NOs: 3 to 24.

In certain embodiments, aptamers of the invention comprise or consist of a nucleic acid sequence comprising at least about 30, 35, 40, 45, 50, 51, 52, 53, 54, 55, 60 or more consecutive nucleotides of a sequence having at least 85%, at least 90%, at least 95%, at least 96%, at least 97%, at least 98%, at least 99% or more identity with SEQ ID NOs: 3 to 19.

In certain embodiments, aptamers of the invention comprise or consist of a nucleic acid sequence comprising at least about 30, 35, 40, 45, 50, 51, 52, 53, 54, 55, 60 or more consecutive nucleotides of a sequence having at least 85%, at least 90%, at least 95%, at least 96%, at least 97%, at least 98%, at least 99% or more identity with SEQ ID NO: 3.

In certain embodiments, aptamers of the invention comprise or consist of a nucleic acid sequence comprising at least about 30, 35, 40, 45, 50, 51, 52, 53, 54, 55, 60 or more consecutive nucleotides of a sequence comprising any one of SEQ ID NOs: 3 to 24 or 27 to 30.

In certain embodiments, aptamers of the invention comprise or consist of a nucleic acid sequence comprising at least about 30, 35, 40, 45, 50, 51, 52, 53, 54, 55, 60 or more consecutive nucleotides of a sequence comprising any one of SEQ ID NOs: 3 to 24.

In certain embodiments, aptamers of the invention comprise or consist of a nucleic acid sequence comprising at least about 30, 35, 40, 45, 50, 51, 52, 53, 54, 55, 60 or more consecutive nucleotides of a sequence comprising any one of SEQ ID NOs: 3 to 19.

In certain embodiments, aptamers of the invention comprise or consist of a nucleic acid sequence comprising at least about 30, 35, 40, 45, 50, 51, 52, 53, 54, 55, 60 or more consecutive nucleotides of a sequence comprising any one of SEQ ID NO: 3.

The aptamers of the invention may comprise natural or non-natural nucleotides and/or base derivatives (or combinations thereof). In certain embodiments, the aptamers comprise one or more modifications such that they comprise a chemical structure other than deoxyribose, ribose, phosphate, adenine (A), guanine (G), cytosine (C), thymine (T), or uracil (U). The aptamers may be modified at the nucleobase, at the sugar or at the phosphate backbone.

In certain embodiments, the aptamers comprise one or more modified nucleotides. Exemplary modifications include for example nucleotides comprising an alkylation, arylation or acetylation, alkoxylation, halogenation, amino group, or another functional group. Examples of modified nucleotides include 2′-fluoro ribonucleotides, 2′-NH 2-, 2′-OCH 3- and 2′-O-methoxyethyl ribonucleotides, which are used for RNA aptamers.

The aptamers of the invention may be wholly or partly phosphorothioate or DNA, phosphorodithioate or DNA, phosphoroselenoate or DNA, phosphorodiselenoate or DNA, locked nucleic acid (LNA), peptide nucleic acid (PNA), N3′-P5′phosphoramidate RNA/DNA, cyclohexene nucleic acid (CeNA), tricyclo DNA (tcDNA) or spiegelmer, or the phosphoramidate morpholine (PMO) components or any other modification known to those skilled in the art (see also Chan et al., Clinical and Experimental Pharmacology and Physiology (2006) 33, 533-540).

Some of the modifications allow the aptamers to be stabilized against nucleic acid-cleaving enzymes. In the stabilization of the aptamers, a distinction can generally be made between the subsequent modification of the aptamers and the selection with already modified RNA/DNA. The stabilization does not affect the affinity of the modified RNA/DNA aptamers but prevents the rapid decomposition of the aptamers in an organism or biological solutions by RNases/DNases. An aptamer is referred to as stabilized in the context of the present invention if the half-life in the sample (e.g. biological medium) is greater than one minute, preferably greater than one hour, more preferably greater than one day. The aptamers may also be modified with reporter molecules which, in addition to the detection of the labelled aptamers, may also contribute to increasing the stability.

Aptamers are characterised by the formation of a specific three-dimensional structure that depends on the nucleic acid sequence. The three-dimensional structure of an aptamer arises due to Watson and Crick intramolecular base pairing, Hoogsteen base pairing (quadruplex), wobble-pair formation or other non-canonical base interactions. This structure enables aptamers, analogous to antigen-antibody binding, to bind target structures accurately. A nucleic acid sequence of an aptamer may, under defined conditions, have a three-dimensional structure that is specific to a defined target structure.

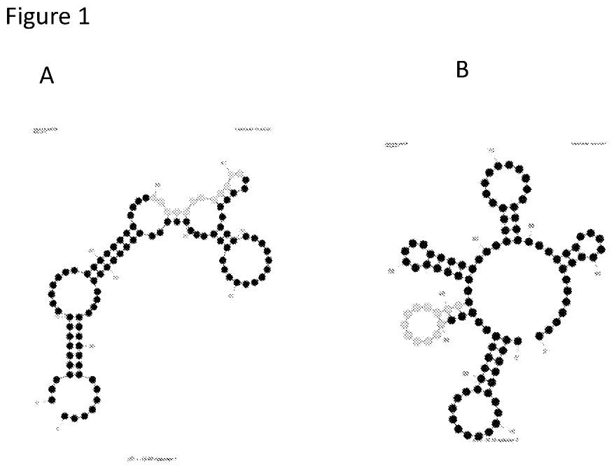

In certain embodiments, the aptamer comprises a secondary structure as shown in FIG. 1. The secondary structure analysis of the aptamers was performed by means of the free-energy minimization algorithm Mfold (M Zuker. Mfold web server for nucleic acid folding and hybridization prediction. Nucleic Acids Res. 31(13), 3406-3415, 2003). In certain embodiments, the aptamers of the invention may contain at least 1, 2, 3, 4, 5, 6, 7, 8, 9, 10 or more nucleotide variations as compared to any one of SEQ ID NOs: 3 to 24 or 27 to 30. Positions where such variations can be introduced can be determined based on, for example, the secondary structures shown in FIG. 1.

The invention also provides aptamers that compete for binding to Imatinib with aptamers as described herein. In certain embodiments, the invention provides aptamers that compete for binding to Imatinib with the aptamers set forth in any one of SEQ ID NOs: 3 to 24 or 27 to 30. In certain embodiments, competition assays may be used identify an aptamer that competes for binding to Imatinib. In an exemplary competition assay, immobilized Imatinib is incubated in a solution comprising a first labelled aptamer that binds to Imatinib and a second unlabelled aptamer that is being tested for its ability to compete with the first aptamer for binding to Imatinib. As a control, immobilized Imatinib may be incubated in a solution comprising the first labelled aptamer but not the second unlabelled aptamer. After incubation under conditions permissive for binding of the first aptamer to Imatinib excess unbound aptamer may be removed, and the amount of label associated with immobilized Imatinib measured. If the amount of label associated with immobilized Imatinib is substantially reduced in the test sample relative to the control sample, then that indicates that the second aptamer is competing with the first aptamer for binding to Imatinib.

### Immobilisation Oligonucleotides

In certain embodiments, aptamers are detected in the absence of any immobilisation oligonucleotide. For example, aptamers of the invention may be immobilised to a support via a linker sequence as described herein.

In certain embodiments, aptamers of the invention comprise an immobilisation region. The immobilisation region of the aptamer may hybridise over at least a portion of a suitably designed immobilisation oligonucleotide.

In certain embodiments, the immobilisation oligonucleotide comprises a nucleic acid sequence which is configured to hybridise to the immobilisation region of the aptamer over at least a portion of its length. For example, the immobilisation oligonucleotide (or portion thereof) may be configured to form a double-stranded duplex structure with the immobilisation region (or portion thereof) of the aptamer.

In certain embodiments, the immobilisation oligonucleotide is between about 10 to about 20 nucleotides in length, e.g. about 10, 11, 12, 13, 14, 15, 16, 17, 18, 19, 20 or more nucleotides in length. Typically, the immobilisation oligonucleotide is complementary to an immobilisation region of the aptamer. In certain embodiments, the immobilisation oligonucleotide is a “universal” oligonucleotide capable of hybridising to immobilisation regions included in a plurality of aptamers.

In certain embodiments, the immobilisation oligonucleotide or aptamer comprises a linker portion with a suitable functional moiety to allow surface attachment of the immobilisation oligonucleotide and/or aptamer. The functional moiety may be selected from biotin, thiol, and amine or any other suitable group known to those skilled in the art.

In certain embodiments, the immobilisation oligonucleotide or aptamer comprises a spacer molecule e.g. a spacer molecule selected from a polynucleotide molecule, C6 spacer molecule, a C12 spacer molecule, another length C spacer molecule, a hexaethylene glycol molecule, a hexanediol, and/or polyethylene glycol. The linker may be for example a biotin linker. In certain embodiments, the immobilisation oligonucleotide or aptamer may be conjugated to streptavidin, avidin and/or neutravidin.

In certain embodiments, the immobilisation oligonucleotide or aptamer may be modified for attachment to the support surface. For example, the immobilisation oligonucleotide or aptamer may be attached via a silane linkage. The immobilisation oligonucleotide or aptamer may be succinylated (e.g. to attach the immobilisation oligonucleotide or aptamer to aminophenyl or aminopropyl-derivatized glass). Aptly, the support is aminophenyl or aminopropyl-derivatized. In certain embodiments, the immobilisation oligonucleotide or aptamer comprises a NH₂ modification (e.g. to attach to epoxy silane or isothiocyanate coated glass). Typically, the support surface is coated with an epoxy silane or isothiocyanate. In certain embodiments, the immobilisation oligonucleotide or aptamer is hydrazide-modified in order to attach to an aldehyde or epoxide molecule.

### Support

In certain embodiments, the aptamer or immobilisation oligonucleotide is attached to a support. Typically, the support is a solid support such as a membrane or a bead. The support may be a two-dimensional support e.g. a microplate or a three-dimensional support e.g. a bead. In certain embodiments, the support may comprise at least one magnetic bead.

In certain embodiments, the support may comprise at least one nanoparticle e.g. gold nanoparticles or the like. In yet further embodiments, the support comprises a microtiter or other assay plate, a strip, a membrane, a film, a gel, a chip, a microparticle, a nanofiber, a nanotube, a micelle, a micropore, a nanopore or a biosensor surface. In certain embodiments, the biosensor surface may be a probe tip surface, a biosensor flow-channel or similar.

In certain embodiments, the aptamer or immobilisation oligonucleotide may be attached, directly or indirectly, to a magnetic bead, which may be e.g. carboxy-terminated, avidin-modified or epoxy-activated or otherwise modified with a compatible reactive group.

Immobilisation of oligonucleotides to a support e.g. a solid phase support can be accomplished in a variety of ways and in any manner known to those skilled in the art for immobilising DNA or RNA on solids. The immobilisation of aptamers on nanoparticles is e.g. as described in WO2005/13817. For example, a solid phase of paper or a porous material may be wetted with the liquid phase aptamer, and the liquid phase subsequently volatilized leaving the aptamer in the paper or porous material.

In certain embodiments, the support comprises a membrane, e.g. a nitrocellulose, a polyethylene (PE), a polytetrafluoroethylene (PTFE), a polypropylene(PP), a cellulose acetate (CA), a polyacrylonitrile (PAN), a polyimide (PI), a polysulfone (PS), a polyethersulfone (PES) membrane or an inorganic membrane comprising aluminium oxide (Al₂O₃), silicon oxide (SiO₂) and/or zirconium oxide (ZrO₂). Particularly suitable materials from which a support can be made include for example inorganic polymers, organic polymers, glasses, organic and inorganic crystals, minerals, oxides, ceramics, metals, especially precious metals, carbon and semiconductors. A particularly suitable organic polymer is a polymer based on polystyrene. Biopolymers, such as cellulose, dextran, agar, agarose and Sephadex, which may be functionalized, in particular as nitrocellulose or cyanogen bromide Sephadex, can be used as polymers which provide a solid support.

### Detectable Labels

In certain embodiments, the aptamers of the invention are used to detect and/or quantify the amount of Imatinib in a sample. Typically, the aptamers comprise a detectable label. Any label capable of facilitating detection and/or quantification of the aptamers may be used herein.

In certain embodiments, the detectable label is a fluorescent moiety, e.g. a fluorescent/quencher compound. Fluorescent/quencher compounds are known in the art. See, for example, Mary Katherine Johansson, Methods in Molecular Biol. 335: Fluorescent Energy Transfer Nucleic Acid Probes: Designs and Protocols, 2006, Didenko, ed., Humana Press, Totowa, N.J., and Marras et al., 2002, Nucl. Acids Res. 30, e122 (incorporated by reference herein).

In certain embodiments, the detectable label is FAM. In certain embodiments, the FAM-label is situated at the first or second primer region of the aptamer. The person skilled in the art would understand that the label could be located at any suitable position within the aptamer. Moieties that result in an increase in detectable signal when in proximity of each other may also be used herein, for example, as a result of fluorescence resonance energy transfer (“FRET”); suitable pairs include but are not limited to fluoroscein and tetramethylrhodamine; rhodamine 6G and malachite green, and FITC and thiosemicarbazole, to name a few.

In certain embodiments, the detectable label is selected from a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

In certain embodiments, the detectable label is a fluorescent protein such as Green Fluorescent Protein (GFP) or any other fluorescent protein known to those skilled in the art.

In certain embodiments, the detectable label is an enzyme. For example, the enzyme may be selected from horseradish peroxidase, alkaline phosphatase, urease, β-galactosidase or any other enzyme known to those skilled in the art.

In certain embodiments, the nature of the detection will be dependent on the detectable label used. For example, the label may be detectable by virtue of its colour e.g. gold nanoparticles. A colour can be detected quantitatively by an optical reader or camera e.g. a camera with imaging software.

In certain embodiments, the detectable label is a fluorescent label e.g. a quantum dot. In such embodiments, the detection means may comprise a fluorescent plate reader, strip reader or similar, which is configured to record fluorescence intensity.

In embodiments in which the detectable label is an enzyme label, the detection means may, for example, be colorimetric, chemiluminescence and/or electrochemical (for example, using an electrochemical detector). Typically, electrochemical sensing is through conjugation of a redox reporter (e.g. methylene blue or ferrocene) to one end of the aptamer and a sensor surface to the other end. Typically, a change in aptamer conformation upon target binding changes the distance between the reporter and sensor to provide a readout.

In certain embodiments, the detectable label may further comprise enzymes such as horseradish peroxidase (HRP), Alkaline phosphatase (APP) or similar, to catalytically turnover a substrate to give an amplified signal.

In certain embodiments, the invention provides a complex (e.g. conjugate) comprising aptamers of the invention and a detectable molecule. Typically, the aptamers of the invention are covalently or physically conjugated to a detectable molecule.

In certain embodiments, the detectable molecule is a visual, optical, photonic, electronic, acoustic, opto-acoustic, mass, electrochemical, electro-optical, spectrometric, enzymatic, or otherwise physically, chemically or biochemically detectable label.

In certain embodiments, the detectable molecule is detected by luminescence, UV/VIS spectroscopy, enzymatically, electrochemically or radioactively. Luminescence refers to the emission of light. For example, photoluminescence, chemiluminescence and bioluminescence are used for detection of the label. In photoluminescence or fluorescence, excitation occurs by absorption of photons. Exemplary fluorophores include, without limitation, bisbenzimidazole, fluorescein, acridine orange, Cy5, Cy3 or propidium iodide, which can be covalently coupled to aptamers, tetramethyl-6-carboxyhodamine (TAMRA), Texas Red (TR), rhodamine, Alexa Fluor dyes (et al. Fluorescent dyes of different wavelengths from different companies).

In certain embodiments, the detectable molecule is a colloidal metallic particle, e.g. gold nanoparticle, colloidal non-metallic particle, quantum dot, organic polymer, latex particle, nanofiber (e.g. carbon nanofiber), nanotube (e.g. carbon nanotube), dendrimer, protein or liposome with signal-generating substances. Colloidal particles can be detected colorimetrically.

In certain embodiments, the detectable molecule is an enzyme. In certain embodiments, the enzyme may convert substrates to coloured products, e.g. peroxidase, luciferase, β-galactosidase or alkaline phosphatase. For example, the colourless substrate X-gal is converted by the activity of β-galactosidase to a blue product whose colour is visually detected.

In certain embodiments, the detection molecule is a radioactive isotope. The detection can also be carried out by means of radioactive isotopes with which the aptamer is labelled, including but not limited to 3H, 14C, 32P, 33P, 35S or 125I, more preferably 32P, 33P or 125I. In the scintillation counting, the radioactive radiation emitted by the radioactively labelled aptamer target complex is measured indirectly. A scintillator substance is excited by the isotope's radioactive emissions. During the transition of the scintillation material, back to the ground state, the excitation energy is released again as flashes of light, which are amplified and counted by a photomultiplier.

In certain embodiments, the detectable molecule is selected from digoxigenin and biotin. Thus, the aptamers may also be labelled with digoxigenin or biotin, which are bound for example by antibodies or streptavidin, which may in turn carry a label, such as an enzyme conjugate. The prior covalent linkage (conjugation) of an aptamer with an enzyme can be accomplished in several known ways. Detection of aptamer binding may also be achieved through labelling of the aptamer with a radioisotope in an RIA (radioactive immunoassay), preferably with 125I, or by fluorescence in a FIA (fluoroimmunoassay) with fluorophores, preferably with fluorescein or FITC.

### Apparatus

The apparatus according to the invention may be provided in a number of different formats. In certain embodiments, the invention provides apparatus for detecting the presence, absence or level of Imatinib in a sample, the apparatus comprising an aptamer as described herein.

In certain embodiments, the apparatus comprises a support as described herein. For example, in the absence of Imatinib, the aptamer may be secured directly or indirectly to a support to immobilise it.

In certain embodiments, the apparatus comprises an immobilisation oligonucleotide as described herein.

In certain embodiments, the aptamer may be attached by way of hybridizing to the immobilisation oligonucleotide which is in turn directly or indirectly attached to the support. Alternatively, the aptamer itself may be attached directly or indirectly (e.g. via a linker) to the support surface. In this embodiment, the immobilisation oligonucleotide is configured to hybridise to at least a portion of the aptamer. In this embodiment, the disruption of the interaction between the immobilisation oligonucleotide and aptamer may be measured as an indirect measurement of the presence of Imatinib.

Certain embodiments of the present invention utilise the ability of the aptamer to change conformation when it binds to Imatinib. The conformational change may cause the aptamer to disassociate from the immobilisation oligonucleotide thus releasing either the immobilisation oligonucleotide or the aptamer in complex with Imatinib depending on which is attached to the support. If Imatinib is not present, the aptamer does not undergo the conformation change and as such remains hybridized to the immobilisation oligonucleotide.

In certain embodiments, the apparatus comprises a linker molecule attached to the support and wherein the linker molecule is configured to hybridize to the aptamer, and further wherein the immobilisation oligonucleotide is configured to hybridize to the aptamer when the aptamer is hybridized to the linker molecule.

Aptly, a linker molecule is attached to the support and wherein the linker molecule is configured to hybridize to the immobilisation oligonucleotide and further wherein the aptamer is configured to hybridize to the immobilisation oligonucleotide when the immobilisation oligonucleotide is hybridized to the linker molecule. In certain embodiments, the linker molecule is a DNA or an RNA molecule or a mixed DNA/RNA molecule, wherein optionally the linker molecule comprises one or more modified nucleotides.

In certain embodiments, the apparatus may be a biosensor. Biosensors are found in many different formats. In certain embodiments, the biosensor comprises the aptamer and a transducer which converts the binding event between the aptamer and Imatinib into an electrically quantifiable signal. The biosensor may be comprised in a vessel or a probe or the like.

In addition, the apparatus may further comprise other elements such as a signal processing device, output electronics, a display device, a data processing device, a data memory device and interfaces to other devices. In certain embodiments, a sample containing Imatinib is brought into contact with the biosensor. Imatinib may then be identified via the changes in the aptamer properties upon specific binding of Imatinib to the aptamer.

The sensitivity of the sensor may be influenced by the transducer used. The transducer converts the signal from the binding event, which is proportional to the concentration of the target molecule in the sample, into an electrically quantifiable measurement signal. Signalling occurs due to the molecular interaction between the aptamer and Imatinib.

With a biosensor according to the invention, qualitative, quantitative and/or semi-quantitative analytical information can be obtained.

The measurement in optical transducers can be based on principles of photometry, whereby, for example, colour or luminescence intensity changes are detected. Optical methods include the measurement of fluorescence, phosphorescence, bioluminescence and chemiluminescence, infrared transitions and light scattering. The optical methods also include the measurement of layer thickness changes when Imatinib is bound to the aptamer. The layer thickness can be measured, for example, by surface plasmon resonance (SPR), reflectometric interference spectroscopy (RIfS), biolayer interferometry (BLI) or similar.

Furthermore, the interference on thin layers (SPR or RIfS) and the change of the evanescent field can be measured. Acoustic transducers use the frequency changes of a piezoelectric quartz crystal, which detects highly sensitive mass changes that occur when target binds to aptamer. The quartz crystal used is placed in an oscillating electric field and the resonant frequency of the crystal is measured. A mass change on the surface of the quartz crystal can be quantified.

In certain embodiments, the apparatus is a BLI (Biolayer Interferometry) apparatus or similar apparatus. BLI is a label-free technology for measuring biomolecular interactions. It is an optical analytical technique that analyses changes in the interference pattern of white light reflected from two surfaces: a layer of immobilised ligand on the biosensor tip, and an internal reference layer. Any change in the number of molecules bound to the biosensor tip causes a shift in the interference pattern that can be measured in real-time. Only molecules binding to or dissociating from the biosensor can shift the interference pattern and generate a response profile on the BLI sensor. Unbound molecules, changes in the refractive index of the surrounding medium, or changes in flow rate do not affect the interference pattern. The Displacement selection principle allows development of detection assays based on the duplex formation between the immobilisation sequence of the aptamers and the immobilisation oligonucleotide. The target-dependent conformational change may lead to the release of the aptamer from the duplex structure. This switch from the hybridised duplex to the displaced stage of the aptamer can be used to generate a recordable signal, target concentration dependent signal.

Depending on the design, qualitative, quantitative and/or semi-quantitative analytical information about the target to be measured can be obtained with the measuring device. The detection means may be for example a portable meter.

The invention also provides a test strip and/or lateral flow device comprising any aptamer or complex as described herein. Lateral flow devices may also be referred to as lateral flow tests, lateral flow assays and lateral flow immunoassays.

In certain embodiments, the lateral flow device comprises a support onto which an immobilisation oligonucleotide is attached. The immobilisation oligonucleotide is configured to hybridise to at least a portion of an immobilisation region of an aptamer as described herein. Any sample as described herein (e.g. a blood or plasma sample) may be introduced. If the sample comprises Imatinib, the aptamer may bind to Imatinib and undergo a conformational change, resulting in the aptamer disassociating from the immobilisation oligonucleotide.

In certain embodiments, the apparatus may be suitable for use in assays such as ELISA (enzyme-linked immunosorbent assay). When aptamers are used in place of antibodies, the resulting assay is often referred to as an “ELONA” (enzyme-linked oligonucleotide assay), “ELASA” (enzyme linked aptamer sorbent assay), “ELAA” (enzyme-linked aptamer assay) or similar. Incorporating aptamers into these ELISA-like assay platforms can result in increased sensitivity, allow a greater number of analytes to be detected; including analytes for which there are no antibodies available and a wide range of outputs, since aptamers can be conjugated to multiple reporter molecules including fluorophores, quencher molecules and/or any other detection moiety as described herein.

In certain embodiments, the apparatus may comprise a vessel. The aptamer specific to Imatinib may be immobilized via hybridization to an immobilisation oligonucleotide in the vessel (e.g. the surface of the vessel). A sample which may contain Imatinib may be added to the vessel. If the sample contains Imatinib, this target may bind to the aptamer resulting in a conformational change which in turn results in displacement of the aptamer from the immobilisation oligonucleotide. The displaced aptamer may then be detected using any suitable method described herein.

### Methods of Detecting Imatinib

In certain embodiments, the invention provides methods for detecting the presence, absence or amount of Imatinib in a sample.

In certain embodiments, the sample is synthetic (e.g. non-biological). For example, the sample may be a pharmaceutical composition comprising (or suspected of comprising) Imatinib. In certain embodiments, the invention provides a method for quantifying the amount of Imatinib during manufacture of a pharmaceutical composition.

In certain embodiments, the sample is biological. For example, the sample may comprise whole blood, leukocytes, peripheral blood mononuclear cells, plasma, serum, sputum, breath, urine, semen, saliva, meningial fluid, amniotic fluid, glandular fluid, lymph fluid, nipple aspirate, bronchial aspirate, synovial fluid, joint aspirate, cells, a cellular extract, stool, tissue, a tissue biopsy or cerebrospinal fluid. Typically, the sample is a blood (e.g. plasma) sample. In certain embodiments, the sample is pre-treated, such as by mixing, addition of enzymes, buffers, salt solutions or markers, or purified.

In certain embodiments, the sample is obtained from a subject undergoing Imatinib therapy. The subject may be any animal (e.g. a cat, dog or horse). Typically, the subject is human. Typically, the subject has or is suspected of having a cancer such as leukemia (e.g. CML or ALL), gastrointestinal stromal tumors (GIST), systemic mastocytosis or myelodysplastic syndrome. Typically, the leukemia is Philadelphia chromosome-positive (PH+).

In the methods for detecting the presence, absence or amount of Imatinib in a sample, the sample is interacted (i.e. contacted) with an aptamer as described herein. For example, the sample and aptamers as described herein may be incubated under conditions sufficient for at least a portion of the aptamer to bind to Imatinib in the sample.

A person skilled in the art will understand that the conditions required for binding to occur between the aptamers described herein and Imatinib. In certain embodiments the sample and aptamer may be incubated at temperatures between about 20 C and about 37 C, preferably about 22 C. In certain embodiments, the sample and aptamer may be diluted to different concentrations (e.g. at least about 1%, 5%, 10%, 20%, 25%, 30%, 40%, 50%, 60%, 70% 80% v/v or more) with appropriate buffers (e.g. PBS or the like). In certain embodiments, the sample and aptamer may be incubated whilst shaking and/or mixing. In certain embodiments, the sample and aptamer are incubated for at least 1 minute, at least 5 minutes, at least 15 minutes, at least 1 hour or more.

In certain embodiments, binding of the aptamer and Imatinib leads to formation of an aptamer-Imatinib complex. The binding or binding event may be detected, for example, visually, optically, photonically, electronically, acoustically, opto-acoustically, by mass, electrochemically, electro-optically, spectrometrically, enzymatically or otherwise chemically, biochemically or physically as described herein.

In certain embodiments, the method comprises interacting the sample with the aptamer of the invention and an immobilisation oligonucleotide as described herein. As discussed above, binding of Imatinib may cause a conformational change in the aptamer resulting in its displacement from the immobilisation oligonucleotide. For example, where the immobilisation oligonucleotide is attached to a support, binding of Imatinib to the aptamer may result in displacement of the aptamer from the support.

In certain embodiments, the binding of the aptamer to the immobilisation oligonucleotide is carried out prior to immobilisation of the immobilisation oligonucleotide to the support. Alternatively, the immobilisation oligonucleotide may be attached to the support prior to hybridization of the nucleic acid molecule to the immobilization oligonucleotide. Either the immobilisation oligonucleotide and/or the nucleic acid molecule may be attached to the support. The attachment may be directly or indirectly e.g. via a linker or other attachment moiety.

The binding of aptamer and Imatinib may be detected using any suitable technique. As discussed above, for example, binding of the aptamer and Imatinib may be detected using a biosensor. In certain embodiments, binding of the aptamer and Imatinib is detected using SPR, RIfS, BLI, LFD or ELONA as described herein.

Advantageously, the aptamers of the invention allow detection of clinically relevant amounts of Imatinib. Typically, the aptamers of the invention have a detection limit of less than about 1 μM Imatinib, e.g. less than about 900 nm, less than about 800 nm, less than about 700 nm, less than about 600 nm or less than about 500 nm Imatinib. Typically, the aptamers of the invention have a detection range from about 0 5 μM to about 10 μM Imatinib, e.g. from about 0.5 to about 5 μM Imatinib. Thus, the aptamers are capable of binding to Imatinib with high specificity and affinity and allow clinical ranges of active Imatinib to be detected in a sample.

### Monitoring of Imatinib During Cancer Treatment

In certain embodiments, the invention provides a method of monitoring the level of Imatinib in a sample obtained from a subject undergoing Imatinib therapy. Thus, the invention provides the opportunity to adjust treatment regime based on the subject's individual needs, allowing more effective and personalised treatment.

In certain embodiments, the invention provides the detection of the amount of Imatinib in a sample obtained from the subject according to any method described herein, followed by treating or preventing cancer in the subject according to the level of Imatinib that is detected.

In certain embodiments, the method comprises administering a dose (e.g. initial dose) of Imatinib to the subject following the detection of the amount of Imatinib in a sample obtained from the subject.

In certain embodiments, the cancer is CML (typically PH+), ALL (typically PH+), GIST, systemic mastocytosis or myelodysplastic syndrome. Typically, the subject is human.

The initial dose of Imatinib may be predicted to be a therapeutically or prophylactically effective amount of Imatinib. Typically, the initial dose of Imatinib is administered orally. The initial dose may be determined according to various parameters, especially the age, weight and condition of the subject to be treated and the required regimen. A physician will be able to determine the required route of administration and dosage for any subject.

In certain embodiments, a newly diagnosed adult or paediatric subject with Ph+ CML or ALL is treated with an initial dose of about 300 to 600 mg Imatinib per day.

In certain embodiments, an adult subject suffering from relapsed or refractory Ph+ CML or ALL is treated with an initial dose of about 400 mg Imatinib per day.

In the methods of treating or preventing cancer, the level of Imatinib in a sample from the subject is detected according to the methods described herein. Typically, the sample is a blood sample. Typically, the plasma trough level (Cmin) of Imatinib in the blood sample is detected (e.g. the lowest concentration reached by Imatinib before the next dose of Imatinib is administered).

If the level of Imatinib is determined to be below a lower threshold level, an increased dose of Imatinib may be administered to the subject. Herein, a “lower threshold level” is understood to mean any plasma level of Imatinib that is considered not likely to lead to tumour response in the subject. For example, the lower threshold level of Imatinib may be about 500 ng/ml or less, about 600 ng/ml or less, about 700 ng/ml or less, about 800 ng/ml or less, about 900 ng/ml or less or about 1000 ng/ml or less.

An “increased dose” is understood to mean a higher dose than the initial dose that acts to increase the level of Imatinib in a further sample to above the lower threshold level (e.g. between about 1000 ng/ml to about 3000 ng/ml). The skilled person would be able to calculate a suitable increased dose, based, for example, on the initial dose of Imatinib and the level of Imatinib in the sample.

If the level of Imatinib is determined to be above an upper threshold level, a decreased dose of Imatinib may be administered to the subject. Herein, an “upper threshold level” is understood to mean any plasma level of Imatinib that is considered likely to lead to toxicity in the subject. For example, the upper threshold level of Imatinib may be about 3000 ng/ml, about 3,500 ng/ml, about 4,000 ng/ml or more.

A “decreased dose” is understood to mean a lower dose than the initial dose that decreases the level of Imatinib in a further sample to between about 1000 ng/ml to about 3000 ng/ml. The skilled person would be able to calculate a suitable decreased dose, based on the initial dose of Imatinib and the level of Imatinib in the sample.

In certain embodiments, the level of Imatinib is detected within 1 week, 2 weeks, 3 weeks, 4 weeks, 2 months, 3 months, 4 months, 5 months, 6 months or 12 months after administering the initial dose of Imatinib to the subject. The level of Imatinib may be detected one or more times, for example at regular intervals after commencing Imatinib treatment. Typically, the level of Imatinib is detected at about 3, 6 and/or 12 months allowing monitoring of the therapeutic levels of Imatinib (and adjusting to target levels if necessary) over the first year of Imatinib treatment.

### Kits

The invention also provides a kit for detecting and/or quantifying Imatinib, wherein the kit comprises one or more aptamers as described herein. Typically, the kit also comprises a detectable molecule as described herein.

In some embodiments, the kit further comprises instructions for use in accordance with any of the methods described herein.

In certain embodiments, the kit further comprises an immobilization sequence, support and/or linker as described herein.

Typically, the kit comprises further components for the reaction intended by the kit or the method to be carried out, for example components for an intended detection of enrichment, separation and/or isolation procedures. Examples are buffer solutions, substrates for a colour reaction, dyes or enzymatic substrates. In the kit, the aptamer may be provided in a variety of forms, for example pre-immobilised onto a support (e.g. solid support), freeze-dried or in a liquid medium.

The kit of the invention may be used for carrying out any method described herein. It will be appreciated that the parts of the kit may be packaged individually in vials or in combination in containers or multi-container units. Typically, manufacture of the kit follows standard procedures which are known to the person skilled in the art.

### EXAMPLES

In the following, the invention will be explained in more detail by means of non-limiting examples of specific embodiments. In the example experiments, standard reagents and buffers free from contamination are used.

### Example 1—Aptamer Selection

Single stranded DNA aptamer selection was performed using the Displacement selection process. Inserted fluorescence markers allowed the quantification of the DNA after different steps of the process by fluorescence measurement.

During the selection process, the ssDNA oligomers of the aptamer library are immobilised onto magnetic beads via a complimentary immobilisation oligonucleotide. After different washing steps to remove unbound and only weakly bound ssDNA molecules, background elution and subsequent target binding steps are performed under the same conditions. Target binding leads to a conformational change of the aptamers. The conformational change causes the aptamers to disassociate from the immobilisation oligonucleotide thus releasing/displacing the aptamer in complex with the target molecule. If the target molecule is not present, the aptamer molecule does not undergo the conformation change and as such remains hybridised to the immobilisation oligonucleotide.

A direct comparison of the amount of unspecific eluted material during the background step and the amount of aptamers which are displaced due to target-binding enables tracking of the selection process. If target-bound material is exponential enriched compared to unspecific background, the aptamer selection process is successful. The enriched aptamer pool can be used as ‘polyclonal aptamer’ or individual aptamer molecules can be isolated from the pool.

Stringency during the selection process was enhanced by introducing counter selection steps. In these steps, the immobilised library is immobilised with unspecific/‘not-wanted’ target molecules to remove ssDNA molecules which have an affinity to these unspecific/‘not-wanted’ targets.

### Aptamer Library and Oligonucleotides

During the selection process, ssDNA oligonucleotide sequences of an aptamer library (manufactured by IDT, Belgium) were immobilised onto magnetic beads via a complimentary immobilisation oligonucleotide (SEQ ID NO: 31).

The nucleotide sequences of the aptamer library have the following structure (in a 5′ to 3′ direction):

P1-R1-I-R2-P2,

wherein P1 is a first primer region, R1 is a first randomized region, I is the immobilisation region, R2 is a further randomized region and P2 is a further primer region wherein at least R1 and/or R2 or a portion thereof are involved in target molecule binding.

The following modified primers were used in the amplification of the oligomers by means of PCR: fluorescein (FAM)-labelled forward primer (P1) with the sequence: 5′-/56FAM/ATCCACGCTCTTTTTCTCC-3′ and PO₄-modified reverse primer (P2) with the sequence: 5′/5Phos/CCTATGTCACCCTCAATGC-3′.

The exemplary biotinylated immobilisation oligonucleotide (I) has the following structure: 5 ‘Bio-GTC-HEGL-GATCGAGCCTCA-3’. All oligonucleotides were chemically synthesised by IDT, Belgium.

The first randomized region (R1) of the library is a sequence of any about 10 nucleic acids. The second randomized region (R2) of the library is a sequence of any about 40 nucleic acids.

Immobilisation of Aptamer Library onto Magnetic Beads

The immobilisation oligonucleotide contains a defined region of 12 nucleotides which is complementary to the immobilisation region of the ssDNA nucleotide sequences of the starting library which enables hybridisation between the sequences. In addition, the immobilisation oligonucleotide carries a 5′ biotin, bound via a hexaethylene glycol (HEGL) residue, which is responsible for the coupling of the immobilisation oligonucleotide to the streptavidin-modified magnetic beads.

For immobilisation, 3 nmol of naive library and 2 nmol of immobilisation oligonucleotide were prehybridised in 250 μL binding buffer ‘BB’ (20 mM Tris-HCl pH 7.4, 100 mM NaCl, 2 mM MgCl₂, 1 mM CaCl₂), 0.01% Tween 20) by heating the mixture for 5 minutes at 95° C. After cooling to 4° C., the pre-hybridised library-immobilisation oligonucleotide mixture was incubated with and thereby immobilised on 10⁹ Dynabeads® M-270 Streptavidin Magnetic Beads (Thermo Fisher Scientific, UK) according to manufacturer's instructions using buffer B&W (5 mM Tris-HCl pH 7.5, 0.5 mM EDTA, 1 M NaCl, 0.01% Tween 20).

From round 2 onwards, 300 pmol of FAM-labelled aptamer library and 200 pmol immobilisation oligonucleotide sequence were hybridised in 100 μL binding buffer (BB) using the same protocol as above. The pre-hybridised aptamer library-immobilisation oligonucleotide mixture was immobilised onto 10⁸ Dynabeads® M-270 Streptavidin Magnetic Beads according to manufacturer's instructions.

### In Vitro Selection Using Displacement Approach

The fluorescence markers incorporated into the aptamer library allow quantification of the aptamer DNA after each step of the process by means of a fluorescence plate reader assay. Fluorescence measurements of fluorescein (FAM)-labelled DNA were conducted using a BMG Fluorescence Plate Reader (FLUOstar OPTIMA, BMG, UK) using the following measuring conditions: excitation 485 nm/emission 520 nm.

Quantification of target displaced and recovered aptamer DNA is based on calculation using calibration curves of FAM-labelled ssDNA (oligonucleotide library) with a range of 0-50 pmol/m L, prepared for each aptamer library in the relevant aptamer selection buffer.

The target Imatinib (Sigma-Aldrich, UK) was diluted to 1 mg/mL (1.7 mM) stock solution in DMSO and stored at −20 C. Working stocks were prepared at 20 μM in selection buffer PBS6 (10 mM Na₂HPO₄/2 mM KH₂PO₄, pH 6.0, 137 mM NaCl, 2.7 mM KCl, 2 mM MgCl₂, 1 mM CaCl₂), 0.01% Tween 20) immediately before usage. The buffers used in the selection of Imatinib targeting aptamers are optimized to improve the efficiency of selection.

Successive rounds of the Displacement Selection process were carried out, comprising the following steps; which are also optimized to reduce interactions with unwanted targets, remove weak binding sequences or sequences which are released through mechanical processes; and improve the efficiency of selection of Imatinib:
Binding of the naïve aptamer library (or the enriched aptamer library prepared from the previous round) to the magnetic beads according to protocol above;
Quantification (fluorescence measurement) of the amount of immobilised aptamer library, input of 500 pmol immobilised naive library into the first selection round, and input of 80 pmol immobilised aptamer library into every following round of selection
Removal of weakly bound oligomers in an elevated temperature wash step at 28° C. for 15 min, in selection buffer PBS6, whilst shaking at 1000 rpm (Thermomixer comfort, Eppendorf, Germany);
Background elution at 22° C. for 45 min, in selection buffer PBS6 whilst shaking at 1000 rpm;
Selection rounds 8, 9 and 10, also included a counter-selection step with 40% human plasma (HUMANPL32NCU2N, BiolVT, UK) in selection buffer PBS6, at 22° C. for 45 min whilst shaking at 1000 rpm;
Target binding at 22° C. for 45 min, in selection buffer PBS6 supplemented with the target molecule (20 μM Imatinib) whilst shaking at 1000 rpm;
After each round of selection, the target-displaced aptamers were separated from the non-displaced aptamers, recovered and directly amplified by semi-asymmetric PCR, using an unequal primer mix (2 μM FAM-labelled forward primer and 0.1 μM PO₄-modified reverse primer);
Double stranded DNA is removed by 30 min. treatment with Lambda exonuclease (EURx, Poland) at 37° C. according manufacturers protocol and the nascent ssDNA is purified using AxyPrep Mag PCR Clean-up Kit (Axygen Biosciences, USA) to obtain an enriched aptamer library. The selected and purified aptamer library was used in the subsequent round of Displacement Selection;
In each round, the amount of aptamer library recovered in the background elution, counter-selection or complex matrix (e.g. plasma) (if applicable) and target binding fractions are quantified by fluorescence measurements. The amount of recovered material in each sample is used to track enrichment of target binding aptamers (relative to background or counter target binders);
In total, 10 rounds of this procedure were performed to enrich Imatinib specific aptamers.

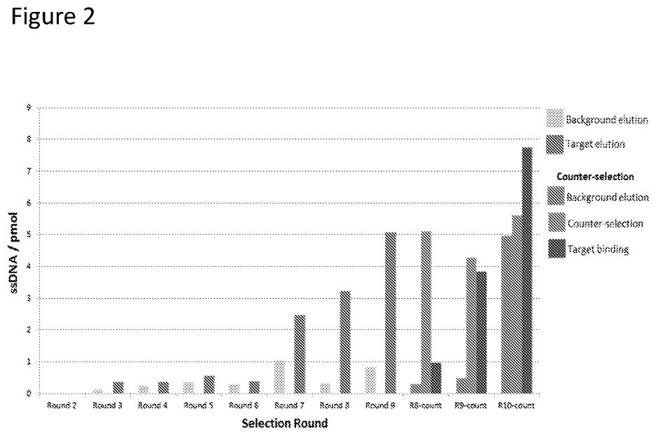

FIG. 2 shows the identification of a high affinity aptamer population. A significant increase in target displacement is observed after round 7, after which counter-selection with human plasma was included. After round 10, a target specific polyclonal population was isolated.

### Construction of a Biosensor and Evaluation of Aptamer-Target Binding

After the 10th round of selection the enriched aptamer population was tested for the binding specificity for the target molecule Imatinib by Biolayer Interferometry (BLI). The experiments were conducted using either the BLItz or Octet QK instruments (ForteBio, Pall Life Sciences, USA).

For aptamer immobilisation onto biosensor probes (Streptavidin-SA Dip & Read Biosensors, ForteBio, Pall Life Sciences, USA), 1.5 μM aptamer (or naïve library) and 1 μM immobilisation oligonucleotide were pre-hybridised in buffer BB by heating the mixture to 95° C. for 10 minutes and immediately cooling to 4° C. for 5 minutes before mixed with an equal volume of 2×B&W buffer (10 mM Tris-HCl pH 7.5, 1 mM EDTA, 2 M NaCl, 0.02% Tween 20). The hybridised oligonucleotides were then immobilised onto streptavidin-coated surfaces, using the biotin group on the immobilisation oligonucleotide. Streptavidin coated probes were incubated with this pre-hybridised mixture for 5 minutes. Three washing steps (30 sec, 120 sec, 30 sec) with buffer PBS6 were performed to remove loosely immobilised library material. The probes were then incubated with target solution (20 μM Imatinib or 20 μM metabolite N-desmethyl Imatinib, in PBS6), for 5 min.

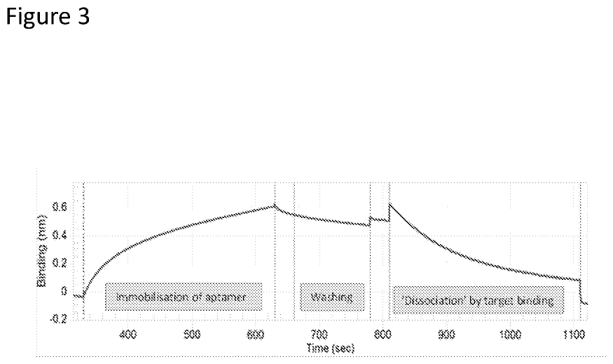

Target binding causes a conformational change in the immobilised aptamer, resulting in aptamer displacement which is seen as decrease in signal (FIG. 3). This “dip and read” BLI assay was used to monitor aptamer-target interactions, identify the best performing aptamer clones and for comparative kinetic analysis.

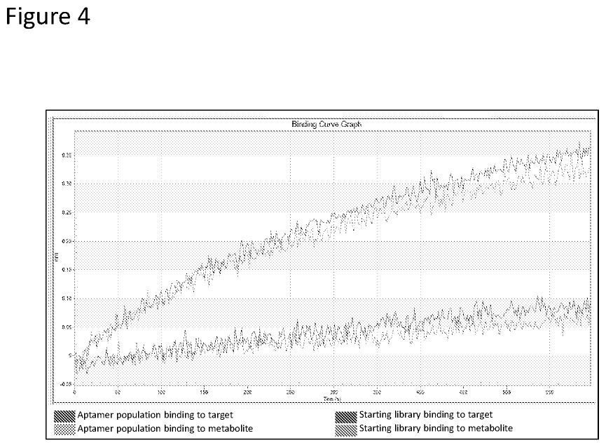

The software of the ForteBio systems (ForteBio Data Analysis 8.0) allows “flipping” the signal to enable comparative kinetic analysis. Using this approach, the BLI binding assays show an improvement in binding of the selected aptamer population to the selection target Imatinib and its main metabolite N-desmethyl Imatinib, in comparison to the starting library (see FIG. 4).

### Cloning

After the last selection round, the recovered aptamer library was amplified by PCR, using unmodified forward and reverse primers. The purified dsDNA was cloned into the pJET1.2/blunt cloning vector, following manufacturers protocol (CloneJET PCR cloning kit, Thermo Fisher Scientic, UK) and used to transform a sequencing strain of E. coli (NEB 5-alpha E. coli C2987H cells) 96 positive transformants/clones were analysed by ‘colony PCR’, using plasmid-specific primers (pJET forward primer and pJET reverse primer, CloneJET PCR cloning kit, Thermo Fisher Scientic, UK.). In parallel, aptamer DNA was produced from the same transformants/clones by ‘aptamer PCR’ using aptamer specific FAM-labelled forward primer and PO₄-modified reverse primer.

### Identification of Individual Aptamers

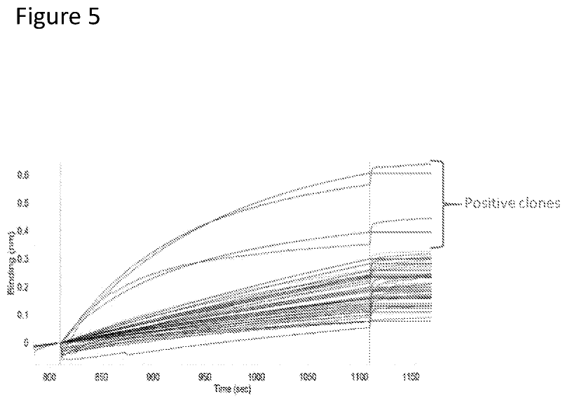

Single stranded DNA was prepared for individual DNA clones according to cloning protocol above. Each clone was then analysed for binding to the target using the BLI assay described above. Two clones showed high affinity to target Imatinib (FIG. 5).

The DNA of aptamer 1 and aptamer 2 clones were sequenced. The obtained sequence data was analysed and aligned by using the web-based tool ClustalW provided by the EBI web server (http://www.ebi.ac.uk/Tools/msa/clustalw2/). The secondary structure analysis of the aptamers was performed using the free-energy minimization algorithm Mfold [[Zuker, M. (2003) Mfold web server for nucleic acid folding and hybridization prediction. Nucleic Acid Res. 31(13), 3406-15](http://mfold.ma.albany.edu/?q=mfold) (FIG. 1).

### Determination of Aptamer Specificity

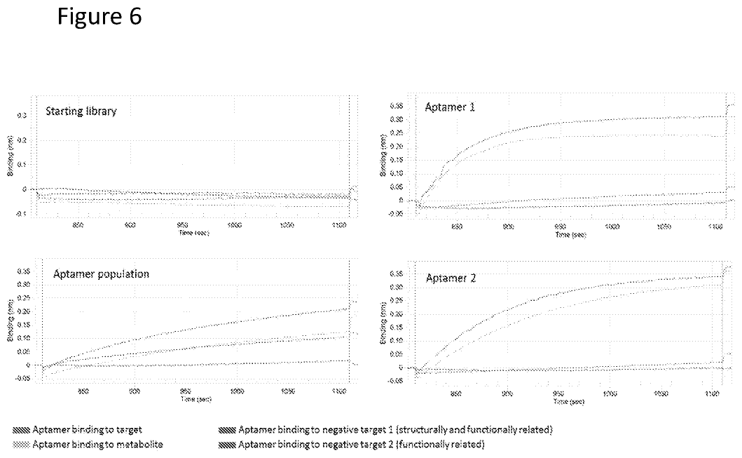

Aptamer specificity was determined using the BLI assay according to protocol described above. Target Imatinib, metabolite N-desmethyl Imatinib, negative target 1 Irinotecan, negative target 2 SN-38 were applied at 10 μM in PBS6 (FIG. 6).

It is clear from FIG. 6 that Aptamer 1 and 2 showed improved binding response over the starting library and the enriched aptamer population from selection round 10, to the selection target and its main metabolite. Other tested small molecule targets (structurally and functionally related) were not bound by either aptamer. Specificity studies were carried out using the BLI Displacement assay (flipped data, buffer subtracted).

### Determination of Aptamer-Imatinib Apparent Binding Affinity

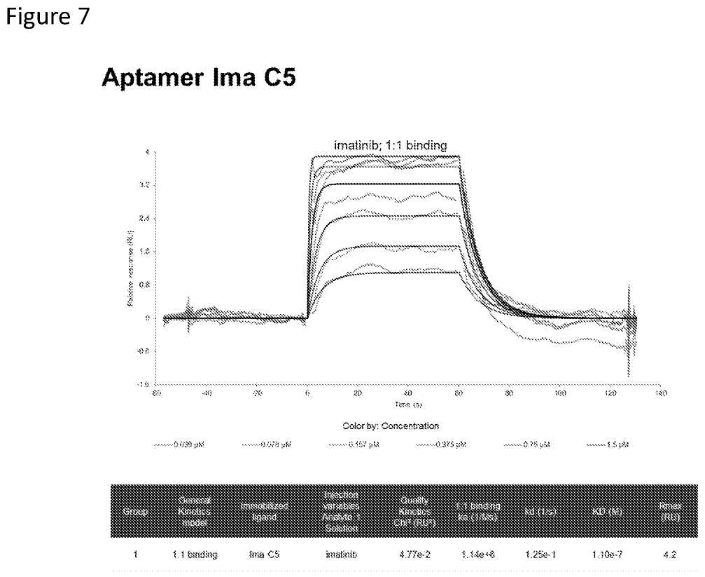

Apparent aptamer affinity was determined by surface plasmon resonance (SPR) using a direct binding assay. A series S CAP chip (GE Healthcare 28920234) was docked, hydrated and preconditioned in a Biacore T200 (GE Healthcare, Uppsala), according to manufacturer's recommendations. The instrument was primed to PBS6 buffer and 5′ biotinylated aptamers were captured on the chip surface following manufacturer's recommendations using a concentration of 1 μM, flow rate of 5 μL/min and a contact time of 10 mins. Full length aptamer 1 (Ima C5), minimal fragment (Ima C5-F6b) and a randomised control were captured on Fc2, Fc3 and Fc4 respectively. Kinetics for Imatinib were determined via multicycle kinetics at 30 μL/min with two blank controls, followed by injection of 0.039, 0.075, 0.157, 0.375, 0.75, 1.5, 3, 6 μM, followed by a blank and duplicate of 0.75 μM with a contact time of 60 s and dissociation time of 60 s. Data was analysed using Biacore Insight evaluation software with a 1:1 binding Langmuir binding model with local RI parameter. The affinity of aptamer 1 (Ima C5) to Imatinib (in PBS6) is calculated with 1.10×10⁻⁷ M (FIG. 7).

### ‘ELISA-Like’ Aptamer Displacement Assay (Microtiter Plate-Based Fluorescence Assay) and Evaluation of Aptamer Selectivity in Human Plasma

For aptamer immobilisation onto streptavidin-coated MTPs (Pierce Streptavidin Coated, HBC, Black 96-Well Plates with SuperBlock Blocking Buffer, Thermo Scientific, USA), 0.75 μM aptamer 1 and 0.5 μM immobilisation oligonucleotide were pre-hybridised in buffer BB by heating the mixture to 95° C. for 10 minutes and immediately cooling to 4° C. for 5 minutes before being mixed with and equal volume of 2×B&W buffer. Microtiter plate MTP 1 was incubated with this pre-hybridisation mixture for 1 h at room temperature while shaking at 1000 rpm on an MTP shaker (IKA Schüttler MTS 4, IKA Werke GmbH & Co. KG, Germany). Immobilisation efficiency was determined by comparing input and output fluorescence pre and post incubation respectively. This allows calculation of the approximate amount of aptamer loaded by fluorescence measurements. The aptamer loaded plate (MTP 1) was extensively washed with selection buffer PBS6 to remove loosely immobilised DNA, before incubated for 1 h at room temperature (1000 rpm at MTP shaker) with a gradient of target Imatinib (10 μM, 5 μM, 2.5 μM, 1.25 μM, 0.625 μM, 0 μM) prepared in buffered human serum (HUMANPL32NCU2N, BiolVT, UK) at 4 concentrations (0%, 10%, 20% & 25% v/v matrix in buffer PBS6). Target-eluted material (from MTP 1) was recovered and amount of target-binding aptamer was determined by fluorescence measurements. Raw data was plotted as ‘fluorescence’ against ‘target concentration’ and at different plasma concentrations.

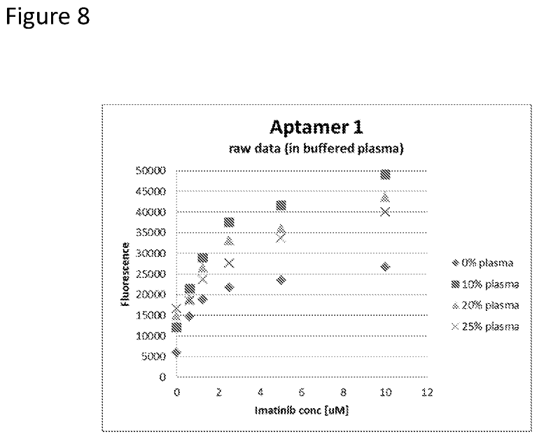

A clear concentration dependent binding to target Imatinib can be observed for aptamer ‘Ima C5’ at all four plasma concentrations, with minimal background binding to the respective concentration of buffered plasma alone (FIG. 8). Tests were carried out at Imatinib concentrations that reflect the therapeutic range of this therapeutic molecule. The limit of detection of Imatinib is less than 1 μM, with clear concentration dependant responses the clinical range for this drug.

### Example 2—Identification of Minimal Effective Binding Fragments of Aptamer 1

For the identification of the minimal functional fragment of aptamer 1, a panel of fragments (truncated versions of the parent aptamer) were produced (manufactured by IDT, Belgium)

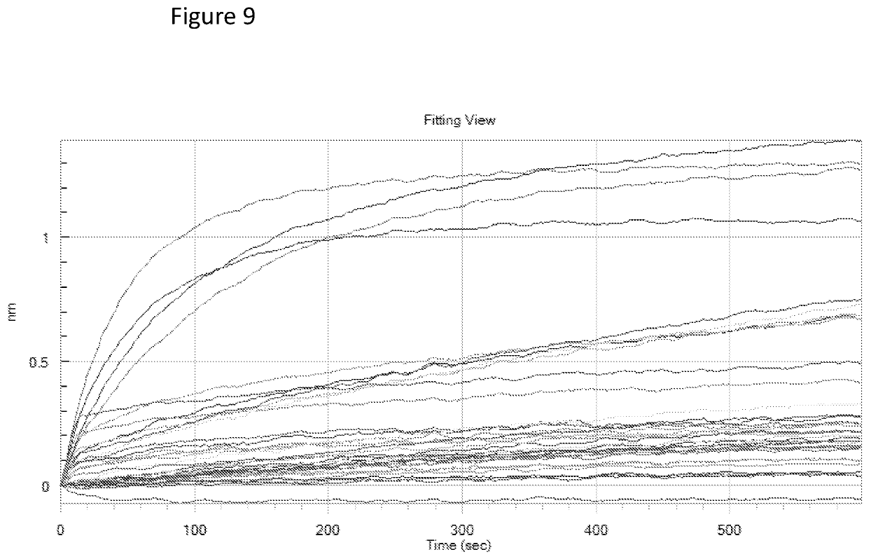

The panel of truncated versions of the parent aptamer 1 was tested for their binding ability to target Imatinib (10 μM in PBS6). In particular, BLI displacement binding studies were used to identify the minimal effective fragments of Aptamer 1. A panel of truncated versions of Aptamer 1 was tested for binding ability to target Imatinib (10 μM in PBS6) (FIG. 9). Minimal fragment identification studies were carried out using the BLI Displacement assay (flipped data, buffer subtracted). Many of the aptamer fragments lose their ability to bind, indicating that the binding site has been removed or compromised. Other fragments show improved binding relative to the parent aptamer (Ima C5). The smallest and best performing aptamer fragment from this panel was found to be fragment F6b (SEQ ID NO: 3).

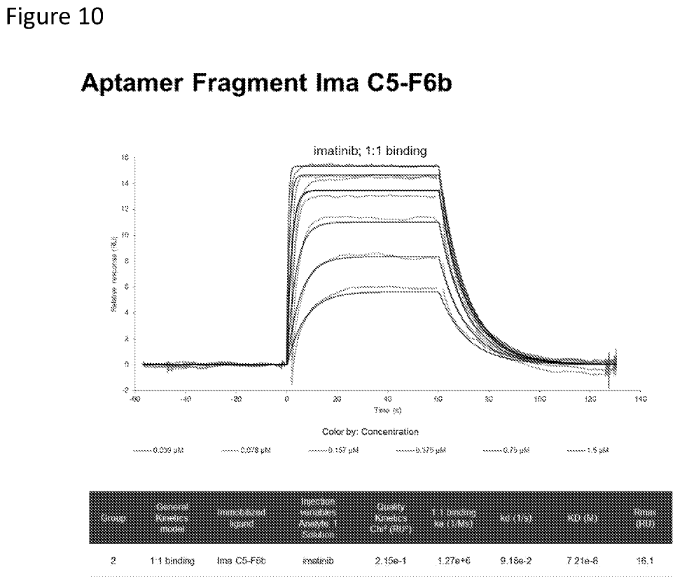

The apparent binding affinity of the minimal effective fragment Ima C5-F6b was tested by SPR using the protocol of the direct binding approach in a Biacore instrument mentioned above. The apparent affinity of aptamer fragment Ima C5-F6b to Imatinib (in PBS6) was calculated with 7.21×10⁻⁸ M (FIG. 10).

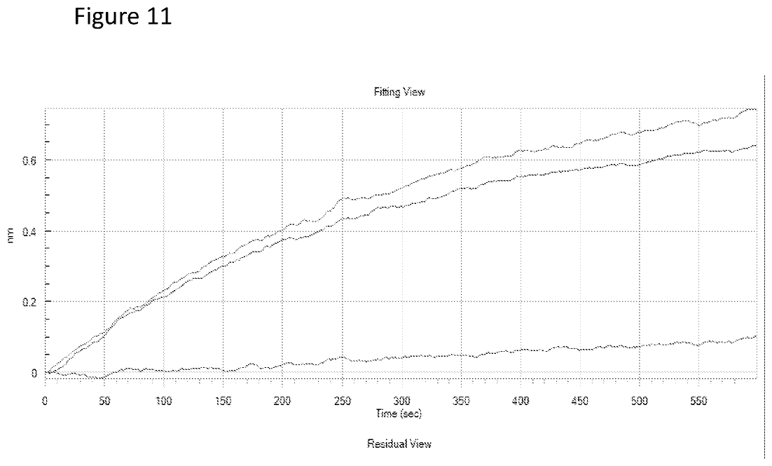

Aptamer specificity was determined using BLI Displacement assay as described above (FIG. 11). Target induced displacement was determined using several related targets and demonstrate that the minimal functional fragment binds to Imatinib and the metabolite N-desmethyl Imatinib, but not to the negative target Irinotecan.

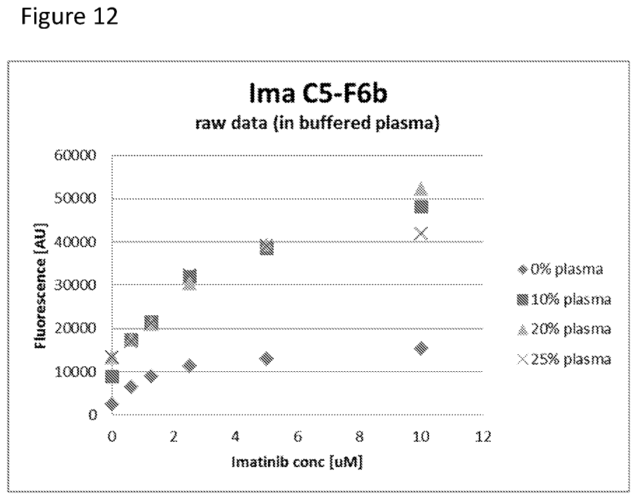

Evaluation of aptamer selectivity in human plasma was verified by ELISA-like Aptamer Displacement Assay (microtiter plate-based fluorescence assay) as described above (FIG. 12). The results show that the minimal functional fragment Ima C5-F6b, is capable of specifically binding to Imatinib in the presence of human plasma with minimal background binding to the plasma alone. Tests were carried out at target concentrations that reflect the therapeutic range of this drug.

The reader's attention is directed to all papers and documents which are filed concurrently with or previous to this specification in connection with this application and which are open to public inspection with this specification, and the contents of all such papers and documents are incorporated herein by reference.

1-27. (canceled)

28. An aptamer capable of specifically binding to Imatinib, comprising:
a nucleic acid sequence selected from any one of SEQ ID NOs: 3 to 24 or 27 to 30;
a nucleic acid sequence having at least 85% identity with any one of SEQ ID NOs: SEQ ID NOs: 3 to 24 or 27 to 30;
a nucleic acid sequence having at least about 30 consecutive nucleotides of any one of SEQ ID NOs 3 to 24 or 27 to 30; or
a nucleic acid sequence having at least about 30 consecutive nucleotides of a sequence having at least 85% identity with any one of SEQ ID Nos 3 to 24 or 27 to 30.

29. The aptamer of claim 28, wherein the aptamer comprises:
(a) a nucleic acid sequence selected from any one of SEQ ID NOs: 3 to 24;
(b) a nucleic acid sequence selected from any one of SEQ ID NOs: 3 to 19;
(c) the nucleic acid sequence of SEQ ID NO:3;
(d) a nucleic acid sequence having at least 95% identity with any one of the sequences of (a) to (c); or
(e) a nucleic acid sequence having at least about 50 consecutive nucleotides of any one of the sequences of (a) to (d).

30. The aptamer of claim 28, wherein the aptamer is a single stranded DNA aptamer.

31. The aptamer of claim 28, wherein the aptamer comprises a detectable label.

32. The aptamer of claim 30, wherein the detectable label is selected from a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

33. The aptamer of claim 28, wherein the aptamer is part of an apparatus comprising a support.

34. The aptamer of claim 33, wherein the support is a bead, a microtiter or other assay plate, a strip, a membrane, a film, a gel, a chip, a microparticle, a nanoparticle, a nanofiber, a nanotube, a micelle, a micropore, a nanopore or a biosensor surface.

35. The aptamer of claim 33, wherein the apparatus comprises an immobilisation oligonucleotide, wherein the immobilisation oligonucleotide comprises a nucleic acid sequence which is at least partially complementary to a nucleic acid sequence of the aptamer and wherein the aptamer is capable of hybridizing to the immobilisation oligonucleotide, optionally wherein the immobilisation oligonucleotide is attached directly or indirectly to the support.

36. The aptamer of claim 33, wherein the aptamer is attached directly or indirectly to the support.

37. The aptamer of claim 33, wherein the apparatus is suitable for surface plasmon resonance (SPR), biolayer interferometry (BLI), lateral flow assay and/or ELONA.

38. A method of detecting the presence, absence or amount of Imatinib in a sample, comprising:
(i) interacting the sample with the aptamer of claim 28; and
(ii) detecting the presence, absence or amount of Imatinib.

39. The method of claim 38, wherein the aptamer is hybridized to an immobilisation oligonucleotide, wherein the immobilisation oligonucleotide comprises a nucleic acid sequence which is at least partially complementary to a nucleic acid sequence of the aptamer, and binding of the aptamer with any Imatinib in the sample leads to displacement of the aptamer and immobilisation oligonucleotide allowing detection of the aptamer.

40. The method of claim 39, wherein the aptamer or immobilisation oligonucleotide is attached to a support.

41. The method of claim 38, wherein the presence, absence or amount of Imatinib is detected by photonic detection, electronic detection, acoustic detection, electrochemical detection, electro-optic detection, enzymatic detection, chemical detection, biochemical detection or physical detection.

42. The method of claim 38, wherein the sample is a synthetic sample, optionally wherein the sample is a pharmaceutical composition containing Imatinib or is obtained from a subject undergoing Imatinib therapy.

43. The method of claim 42, wherein the sample is obtained from a subject undergoing Imatinib therapy and the sample is a blood sample, optionally wherein the plasma trough level (Cₘᵢₙ) of Imatinib is detected.

44. A method of treating or preventing cancer in a subject, comprising:
administering an initial dose of Imatinib to the subject;
detecting the amount of Imatinib in a sample obtained from the subject according to a method as described in claim 38; and
if the level of Imatinib is below a lower threshold level, administering an increased dose of Imatinib to the subject; or
if the level of Imatinib is above an upper threshold level, administering a decreased dose of Imatinib to the subject.

45. The method of claim 44, wherein the sample is a blood sample, optionally wherein the plasma trough level (Cₘᵢₙ) of Imatinib in the blood sample is detected.

46. The method of claim 44, wherein the lower threshold level is about 1000 ng/ml or less and/or the upper threshold level is about 3000 ng/ml or more.

47. The method of claim 44, wherein the level of Imatinib is detected about 3, about 6 and/or about 12 months after administering the initial dose of Imatinib to the subject.
